(19) United States
(12) Patent Application Publication
(10) Pub. No.: US 2025/0019644 A1
(43) Pub. Date: Jan. 16, 2025

(54) Quantitatively Mapping Amino Acid Properties (QMAP) Cell Culture Media

(71) Applicant: UNIVERSITY OF IOWA RESEARCH FOUNDATION, Iowa City, IA (US)

(72) Inventors: Eric TAYLOR, Iowa City, IA (US); Jane BUCHANAN, Iowa City, IA (US)

(73) Assignee: UNIVERSITY OF IOWA RESEARCH FOUNDATION, Iowa City, IA (US)

(21) Appl. No.: 18/767,317

(22) Filed: Jul. 9, 2024

Related U.S. Application Data
(60) Provisional application No. 63/525,758, filed on Jul. 10, 2023.

(51) Int. Cl. C12N 5/00

(52) U.S. Cl. CPC C12N 5/0018; C12N 2500/14; C12N 2500/16; C12N 2500/22; C12N 2500/24; C12N 2500/32; C12N 2500/34; C12N 2500/35; C12N 2500/38; C12N 2500/84

(57) ABSTRACT

In certain embodiments, the present invention provides Qualitatively Mapping Amino Acid Properties (QMAP) cell culture media comprising an amino acid component, wherein the amino acids are present in the component at uniform concentrations; an inorganic salt component; an RPMI 1640 Vitamin Solution component; and auxiliary ingredient component. In certain aspects, the present invention also provides methods of making the QMAP cell culture media and using the QMAP cell culture media to culture cells.

### CROSS-REFERENCE TO RELATED APPLICATION

This application claims priority to U.S. Provisional Application No. 63/525,758, that was filed on Jul. 10, 2023. The entire content of the application referenced above is hereby incorporated by reference.

### STATEMENT REGARDING FEDERALLY SPONSORED RESEARCH

This invention was made with government support under DK104998 awarded by National Institutes of Health. The government has certain rights in the invention.

### BACKGROUND

Cell culture techniques comprising amino acid feeds have a long history of use in studying cultured cells Amino acids are biosynthetic precursors, energy sources, osmolytes and the like, and their use in production cultures strongly correlates with continuous cell growth and productivity. Amino acids are a major class of nutrients that are investigated in hundreds (maybe thousands) of studies per year to understand their role in health and disease processes. Because cell culture studies provide a simplified environment where conditions can be tightly defined and controlled, cell culture models of amino acid function are critical to understanding the role of amino acids in biological systems.

Accordingly, there is a need in the art for medium and methods for culturing cells. wherein the medium allows for controlled conditions in which to grow and study target cells.

### SUMMARY

The inventors have made the surprising discovery that the inclusion of all amino acids at the same concentration (plus other ingredients to support cell growth and viability) in a chemically defined, custom culture media, as opposed to in amounts that replicate specific physiologic milieus is beneficial for quantitatively studying the regulatory functions of individual amino acids in cells.

In one aspect, provided herein is a Qualitatively Mapping Amino Acid Properties (QMAP) cell culture media composition comprising:

(a) an amino acid component comprising glycine, alanine, arginine, aspartic acid, cysteine, glutamic acid, histidine, isoleucine, leucine, lysine, methionine, phenylalanine, proline, serine, threonine, tryptophan, tyrosine, valine, and optionally glutamine and/or asparagine;
wherein the amino acids are present in the component at uniform concentrations;

(b) an inorganic salt component comprising Calcium Chloride (CaCl₂ anhydrous), Magnesium Sulfate (MgSO₄ anhydrous), Potassium Chloride (KCl), Sodium Bicarbonate (NaHCO₃), Sodium Chloride (NaCl), and/or Sodium Phosphate monobasic (NaH₂PO₄—H₂O);

(c) an RPMI 1640 Vitamin Solution component comprising Biotin, Choline chloride, D-Calcium pantothenate, Folic Acid (folate), Niacinamide, Pyridoxine hydrochloride, Riboflavin, Thiamine hydrochloride, and/or Vitamin B12, and optionally Ammonium chloride, i-Inositol, and/or Para-aminobenzoic acid (PABA);

(d) auxiliary ingredient component comprising D-Glucose, Sodium pyruvate, 3-Hydroxybutyric acid sodium salt, L-Carnitine hydrochloride, Sodium L-lactate, Penicillin-streptomycin, and/or Dialyzed fetal bovine serum.

In certain aspects the QMAP cell culture media further comprises (e) Minerals and metals. The specific minerals and metals included depend on the needs of the cells being cultured and what of them remained in the protein bound complement of dialyzed fetal bovine serum. This is determined by one of skill in the art. Exemplary minerals and metals include (in no specific order) iron, zinc, copper, selenium, molybdenum, vanadium, manganese, chromium, cobalt, and possibly others, each in different possible formulations.

In one aspect, provided herein is a method of preparing a QMAP cell culture media comprising:

(a) adding a salt solution consisting of 10× Sodium Bicarbonate (NaHCO₃), Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O) to a sterile container;

(b) adding to the salt solution in the sterile container
(i) RPMI 1640 vitamin solution.
(ii) glucose,
(iii) L-carnitine,
(iv) an amino acid solution consisting of glycine, alanine, arginine, aspartic acid, cysteine, glutamic acid, histidine, isoleucine, leucine, lysine, methionine, phenylalanine, proline, serine, threonine, tryptophan, tyrosine, valine, wherein the amino acid solution comprises a uniform concentration of all the amino acids;
(v) glutamine solution, wherein the glutamine is present at the same concentration as the amino acids in (v);
(vi) asparagine solution, wherein the asparagine is present at the same concentration as the amino acids in (v);
(vii) optionally adding antibiotic, sodium pyruvate, and/or sodium lactate, 3-Hydroxybutyric acid sodium salt, and/or minerals and metals;

(c) adding water to bring media to predetermined volume to form an intermediate media;

(d) adjusting the pH of the intermediate media to about 7.4±0.5; and

(e) sterilizing the intermediate media through 0.22 μm filter, and

(f) adding dialyzed fetal bovine serum to form the QMAP cell culture media.

In one aspect, provided herein is a method of preparing a QMAP cell culture media comprising:

(a) adding to a sterile container
(i) Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O);
(ii) RPMI 1640 vitamin solution,
(iii) glucose,
(iv) L-carnitine,
(v) an amino acid solution consisting of glycine, alanine, arginine, aspartic acid, cysteine, glutamic acid, histidine, isoleucine, leucine, lysine, methionine, phenylalanine, proline, serine, threonine, tryptophan, tyrosine, valine, wherein the amino acid solution comprises a uniform concentration of all the amino acids;
(vi) glutamine solution, wherein the glutamine is present at the same concentration as the amino acids in (v);
(vii) asparagine solution, wherein the asparagine is present at the same concentration as the amino acids in (v),
(viii) optionally adding antibiotic, sodium pyruvate, and/or sodium lactate, 3-Hydroxybutyric acid sodium salt, and/or minerals and metals;

(b) adding water to bring media to predetermined volume to form an intermediate media;

(c) adjusting the pH of the intermediate media to about 74±0.5; and

(d) adding a salt solution consisting of 10× Sodium Bicarbonate (NaHCO₃) to a sterile container to form a secondary media;

(e) sterilizing the secondary media through 0.22 μm filter; and

(f) adding dialyzed fetal bovine serum to form the QMAP cell culture media.

In one aspect, provided herein is a QMAP cell culture media made by the method described above.

In one aspect, provided herein is a method for cultivating a cell in vitro, comprising:

(a) providing QMAP cell culture media described above; and

(b) propagating or maintaining the cell in the QMAP cell culture media to form a cell culture.

In certain aspects, provided herein is a Qualitatively Mapping Amino Acid Properties (QMAP) cell culture media composition comprising:

(a) an amino acid component comprising glycine, alanine, arginine, aspartic acid, cysteine, glutamic acid, histidine, isoleucine, leucine, lysine, methionine, phenylalanine, proline, serine, threonine, tryptophan, tyrosine, valine, and optionally glutamine and/or asparagine; wherein the amino acids are present in the component at uniform concentrations;

(b) an inorganic salt component comprising Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O);

(c) an RPMI 1640 Vitamin Solution component comprising Biotin, Choline chloride, D-Calcium pantothenate, Folic Acid (folate), Niacinamide, Pyridoxine hydrochloride, Riboflavin, Thiamine hydrochloride, and/or Vitamin B12,; and optionally Ammonium chloride, i-Inositol and/or Para-aminobenzoic acid (PABA);

(d) auxiliary ingredient component comprising D-Glucose, Sodium pyruvate, 3-Hydroxybutyric acid sodium salt, L-Carnitine hydrochloride, Sodium L-lactate, Penicillin-streptomycin, and/or Dialyzed fetal bovine serum. and

(e) optionally, minerals and metals.

In certain aspects, provided herein is a method of preparing a QMAP cell culture media comprising:

(a) adding a salt solution consisting of Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O) to a sterile container;

(b) adding to the salt solution in the sterile container
(i) RPMI 1640 vitamin solution,
(ii) glucose,
(iii) L-carnitine,
(iv) an amino acid solution consisting of glycine, alanine, arginine, aspartic acid, cysteine, glutamic acid, histidine, isoleucine, leucine, lysine, methionine, phenylalanine, proline, serine, threonine, tryptophan, tyrosine, valine, wherein the amino acid solution comprises a uniform concentration of all the amino acids,
(v) glutamine solution, wherein the glutamine is present at the same concentration as the amino acids in (v),
(vi) asparagine solution, wherein the asparagine is present at the same concentration as the amino acids in (v);
(vii) optionally adding antibiotic, sodium pyruvate, sodium lactate, 3-Hydroxybutyric acid sodium salt, and/or minerals and metals;

(c) adding water to bring media to predetermined volume to form an intermediate media;

(d) adjusting the pH of the intermediate media to about 74±0.5;

(e) sterilizing the intermediate media through 0.22 μm filter; and

(f) adding dialyzed fetal bovine serum to form the QMAP cell culture media

In certain aspects, provided herein is a method of preparing a QMAP cell culture media comprising:

(a) adding to a sterile container
(i) Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O)
(ii) RPMI 1640 vitamin solution,
(iii) glucose,
(iv) L-carnitine,
(v) an amino acid solution consisting of glycine, alanine, arginine, aspartic acid, cysteine, glutamic acid, histidine, isoleucine, leucine, lysine, methionine, phenylalanine, proline, serine, threonine, tryptophan, tyrosine, valine, wherein the amino acid solution comprises a uniform concentration of all the amino acids,
(vi) glutamine solution, wherein the glutamine is present at the same concentration as the amino acids in (v), (vii) asparagine solution, wherein the asparagine is present at the same concentration as the amino acids in (v):
(viii) optionally adding antibiotic, sodium pyruvate, sodium lactate, and/or minerals and metals;

(b) adding water to bring media to predetermined volume to form an intermediate media;

(c) adjusting the pH of the intermediate media to about 74±0.5; and

(d) adding a salt solution consisting CaCl to a sterile container to form a secondary media;

(e) sterilizing the secondary media through 0.22 μm filter; and

(f) adding dialyzed fetal bovine serum to form the QMAP cell culture media.

In certain aspects, provided herein is a Qualitatively Mapping Amino Acid Properties (QMAP) cell culture media composition comprising:

(a) a pool of amino acids,

wherein the pool of amino acids consists of:
(1) alanine, arginine, aspartic acid, cysteine, glutamic acid, histidine, isoleucine, leucine, lysine, methionine, phenylalanine, proline, serine, threonine, tryptophan, tyrosine, valine, glutamine and asparagine;
(2) glycine, arginine, aspartic acid, cysteine, glutamic acid, histidine, isoleucine, leucine, lysine, methionine, phenylalanine, proline, serine, threonine, tryptophan, tyrosine, valine, glutamine and asparagine;
(3) glycine, alanine, aspartic acid, cysteine, glutamic acid, histidine, isoleucine, leucine, lysine, methionine, phenylalanine, proline, serine, threonine, tryptophan, tyrosine, valine, glutamine and asparagine;
(4) glycine, alanine, arginine, cysteine, glutamic acid, histidine, isoleucine, leucine, lysine, methionine, phenylalanine, proline, serine, threonine, tryptophan, tyrosine, valine, glutamine and asparagine;
(5) glycine, alanine, arginine, aspartic acid, glutamic acid, histidine, isoleucine, leucine, lysine, methionine, phenylalanine, proline, serine, threonine, tryptophan, tyrosine, valine, glutamine and asparagine;
(6) glycine, alanine, arginine, aspartic acid, cysteine, histidine, isoleucine, leucine, lysine, methionine, phenylalanine, proline, serine, threonine, tryptophan, tyrosine, valine, glutamine and asparagine;
(7) glycine, alanine, arginine, aspartic acid, cysteine, glutamic acid, isoleucine, leucine, lysine, methionine, phenylalanine, proline, serine, threonine, tryptophan, tyrosine, valine, glutamine and asparagine;
(8) glycine, alanine, arginine, aspartic acid, cysteine, glutamic acid, histidine, leucine, lysine, methionine, phenylalanine, proline, serine, threonine, tryptophan, tyrosine, valine, glutamine and asparagine;
(9) glycine, alanine, arginine, aspartic acid, cysteine, glutamic acid, histidine, isoleucine, lysine, methionine, phenylalanine, proline, serine, threonine, tryptophan, tyrosine, valine, glutamine and asparagine;
(10) glycine, alanine, arginine, aspartic acid, cysteine, glutamic acid, histidine, isoleucine, leucine, methionine, phenylalanine, proline, serine, threonine, tryptophan, tyrosine, valine, glutamine and asparagine;
(11) glycine, alanine, arginine, aspartic acid, cysteine, glutamic acid, histidine, isoleucine, leucine, lysine, phenylalanine, proline, serine, threonine, tryptophan, tyrosine, valine, glutamine and asparagine;
(12) glycine, alanine, arginine, aspartic acid, cysteine, glutamic acid, histidine, isoleucine, leucine, lysine, methionine, proline, serine, threonine, tryptophan, tyrosine, valine, glutamine and asparagine;
(13) glycine, alanine, arginine, aspartic acid, cysteine, glutamic acid, histidine, isoleucine, leucine, lysine, methionine, phenylalanine, serine, threonine, tryptophan, tyrosine, valine, glutamine and asparagine;
(14) glycine, alanine, arginine, aspartic acid, cysteine, glutamic acid, histidine, isoleucine, leucine, lysine, methionine, phenylalanine, proline, threonine, tryptophan, tyrosine, valine, glutamine and asparagine;
(15) glycine, alanine, arginine, aspartic acid, cysteine, glutamic acid, histidine, isoleucine, leucine, lysine, methionine, phenylalanine, proline, serine, tryptophan, tyrosine, valine, glutamine and asparagine;
(16) glycine, alanine, arginine, aspartic acid, cysteine, glutamic acid, histidine, isoleucine, leucine, lysine, methionine, phenylalanine, proline, serine, threonine, tyrosine, valine, glutamine and asparagine;
(17) glycine, alanine, arginine, aspartic acid, cysteine, glutamic acid, histidine, isoleucine, leucine, lysine, methionine, phenylalanine, proline, serine, threonine, tryptophan, valine, glutamine and asparagine;
(18) glycine, alanine, arginine, aspartic acid, cysteine, glutamic acid, histidine, isoleucine, leucine, lysine, methionine, phenylalanine, proline, serine, threonine, tryptophan, tyrosine, glutamine and asparagine;
(19) glycine, alanine, arginine, aspartic acid, cysteine, glutamic acid, histidine, isoleucine, leucine, lysine, methionine, phenylalanine, proline, serine, threonine, tryptophan, tyrosine, valine, and asparagine;
(20) glycine, alanine, arginine, aspartic acid, cysteine, glutamic acid, histidine, isoleucine, leucine, lysine, methionine, phenylalanine, proline, serine, threonine, tryptophan, tyrosine, valine, and glutamine;

wherein the amino acids are present in the pool at uniform concentrations;

(b) an inorganic salt component comprising Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O);

(c) an RPMI 1640 Vitamin Solution component comprising Biotin, Choline chloride, D-Calcium pantothenate, Folic Acid (folate), Niacinamide, Pyridoxine hydrochloride, Riboflavin, Thiamine hydrochloride, and/or Vitamin B12,; and optionally Ammonium chloride, i-Inositol and/or Para-aminobenzoic acid (PABA);

(d) auxiliary ingredient component comprising D-Glucose, Sodium pyruvate, 3-Hydroxybutyric acid sodium salt, L-Carnitine hydrochloride, Sodium L-lactate, Penicillin-streptomycin, and/or Dialyzed fetal bovine serum. and (e) optionally, minerals and metals.

In certain aspects, provided herein is a Qualitatively Mapping Amino Acid Properties (QMAP) cell culture media composition comprising:

(a) a pool of amino acids,

wherein the pool of amino acids consists of:
(1) alanine, arginine, aspartic acid, cysteine, glutamic acid, histidine, isoleucine, leucine, lysine, methionine, phenylalanine, proline, serine, threonine, tryptophan, tyrosine, valine, glutamine and asparagine;
(2) glycine, arginine, aspartic acid, cysteine, glutamic acid, histidine, isoleucine, leucine, lysine, methionine, phenylalanine, proline, serine, threonine, tryptophan, tyrosine, valine, glutamine and asparagine;
(3) glycine, alanine, aspartic acid, cysteine, glutamic acid, histidine, isoleucine, leucine, lysine, methionine, phenylalanine, proline, serine, threonine, tryptophan, tyrosine, valine, glutamine and asparagine;
(4) glycine, alanine, arginine, cysteine, glutamic acid, histidine, isoleucine, leucine, lysine, methionine, phenylalanine, proline, serine, threonine, tryptophan, tyrosine, valine, glutamine and asparagine;
(5) glycine, alanine, arginine, aspartic acid, glutamic acid, histidine, isoleucine, leucine, lysine, methionine, phenylalanine, proline, serine, threonine, tryptophan, tyrosine, valine, glutamine and asparagine;
(6) glycine, alanine, arginine, aspartic acid, cysteine, histidine, isoleucine, leucine, lysine, methionine, phenylalanine, proline, serine, threonine, tryptophan, tyrosine, valine, glutamine and asparagine;
(7) glycine, alanine, arginine, aspartic acid, cysteine, glutamic acid, isoleucine, leucine, lysine, methionine, phenylalanine, proline, serine, threonine, tryptophan, tyrosine, valine, glutamine and asparagine;
(8) glycine, alanine, arginine, aspartic acid, cysteine, glutamic acid, histidine, leucine, lysine, methionine, phenylalanine, proline, serine, threonine, tryptophan, tyrosine, valine, glutamine and asparagine;
(9) glycine, alanine, arginine, aspartic acid, cysteine, glutamic acid, histidine, isoleucine, lysine, methionine, phenylalanine, proline, serine, threonine, tryptophan, tyrosine, valine, glutamine and asparagine;
(10) glycine, alanine, arginine, aspartic acid, cysteine, glutamic acid, histidine, isoleucine, leucine, methionine, phenylalanine, proline, serine, threonine, tryptophan, tyrosine, valine, glutamine and asparagine;
(11) glycine, alanine, arginine, aspartic acid, cysteine, glutamic acid, histidine, isoleucine, leucine, lysine, phenylalanine, proline, serine, threonine, tryptophan, tyrosine, valine, glutamine and asparagine;
(12) glycine, alanine, arginine, aspartic acid, cysteine, glutamic acid, histidine, isoleucine, leucine, lysine, methionine, proline, serine, threonine, tryptophan, tyrosine, valine, glutamine and asparagine;
(13) glycine, alanine, arginine, aspartic acid, cysteine, glutamic acid, histidine, isoleucine, leucine, lysine, methionine, phenylalanine, serine, threonine, tryptophan, tyrosine, valine, glutamine and asparagine;
(14) glycine, alanine, arginine, aspartic acid, cysteine, glutamic acid, histidine, isoleucine, leucine, lysine, methionine, phenylalanine, proline, threonine, tryptophan, tyrosine, valine, glutamine and asparagine;
(15) glycine, alanine, arginine, aspartic acid, cysteine, glutamic acid, histidine, isoleucine, leucine, lysine, methionine, phenylalanine, proline, serine, tryptophan, tyrosine, valine, glutamine and asparagine;
(16) glycine, alanine, arginine, aspartic acid, cysteine, glutamic acid, histidine, isoleucine, leucine, lysine, methionine, phenylalanine, proline, serine, threonine, tyrosine, valine, glutamine and asparagine;
(17) glycine, alanine, arginine, aspartic acid, cysteine, glutamic acid, histidine, isoleucine, leucine, lysine, methionine, phenylalanine, proline, serine, threonine, tryptophan, valine, glutamine and asparagine;
(18) glycine, alanine, arginine, aspartic acid, cysteine, glutamic acid, histidine, isoleucine, leucine, lysine, methionine, phenylalanine, proline, serine, threonine, tryptophan, tyrosine, glutamine and asparagine;
(19) glycine, alanine, arginine, aspartic acid, cysteine, glutamic acid, histidine, isoleucine, leucine, lysine, methionine, phenylalanine, proline, serine, threonine, tryptophan, tyrosine, valine, and asparagine;
(20) glycine, alanine, arginine, aspartic acid, cysteine, glutamic acid, histidine, isoleucine, leucine, lysine, methionine, phenylalanine, proline, serine, threonine, tryptophan, tyrosine, valine, and glutamine;

wherein the amino acids are present in the pool at uniform concentrations;

(b) an inorganic salt component comprising Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O);

(c) an RPMI 1640 Vitamin Solution component comprising Biotin, Choline chloride, D-Calcium pantothenate, Folic Acid (folate), Niacinamide, Pyridoxine hydrochloride, Riboflavin, Thiamine hydrochloride, and/or Vitamin B12,; and optionally Ammonium chloride, i-Inositol and/or Para-aminobenzoic acid (PABA);

(d) auxiliary ingredient component comprising D-Glucose, Sodium pyruvate, 3-Hydroxybutyric acid sodium salt, L-Carnitine hydrochloride, Sodium L-lactate, Penicillin-streptomycin, and/or Dialyzed fetal bovine serum. and

(e) optionally, minerals and metals.

### BRIEF DESCRIPTION OF DRAWINGS

The patent or application file contains at least one drawing executed in color. Copies of this patent or patent application publication with color drawings will be provided by the Office upon request and payment of the necessary fee.

FIG. 1. QMAP Media Preparation Protocol.

FIGS. 2A-2D. (FIG. 2A) Amino acids regulate metabolism through their roles as material or signaling information. (FIG. 2B) Schematic of the experimental design showing how stable isotope tracing (screen 1) and metabolic profiling (screen 2) are used to determine whether an amino acid acts as material or signaling information or both or neither for a metabolite. (FIG. 2C) Media dilution experiment: Fold changes of essential amino acids (EAAs) relative to the 50:50 DMEM: QMAP media tend to scale linearly with increasing proportions of QMAP. Non-essential amino acids (NEAAs) tend to scale linearly only when a large differential in the NEAA concentrations is present between DMEM and QMAP. (FIG. 2D) Dosing cells with 0.5, 1, and 2 mM of each amino acid results in the linear scaling of intracellular amino acids levels. All data shown is from HCT116 cells (n=3 for (FIG. 2C), n=5 for (FIG. 2D)).

FIGS. 3A-3F. (FIG. 3A) Rank order of ¹³C amino acids according to the number of metabolites that the amino acid provided with ¹³C. (FIG. 3B) Rank order of ¹⁵N amino acids according to the number of metabolites that the amino acid provided with ¹⁵N. (FIG. 3C) Heat map showing the percentage of the metabolite pool (y-axis) that was labeled with ¹³C when cells were treated with ¹³C amino acids (x-axis). (FIG. 3D) Heat map showing the percentage of the metabolite pool (y-axis) that was labeled with ¹⁵N when cells were treated with ¹⁵N amino acids (x-axis). (FIG. 3E) The log2 fold change of intracellular amino acid levels (y-axis) when cells were treated with 0.5, 1, and 2 mM amino acids (x-axis). (FIG. 3F) The log2 fold change of TCA cycle metabolites (y-axis) when cells were treated with 0.5, 1, and 2 mM amino acids (x-axis). All data shown are from HCT116 cells (n=5).

FIG. 4. A specific example of an amino acid acting as information to regulate metabolism. The bubble plot shows the log2 fold change of TCA cycle metabolites when dosed with 0.5, 1, or 2 mM tryptophan. The schematic shows how inhibition of glutaminase by tryptophan or its metabolites could lead to an increase in glutamine and a decrease in distal TCA cycle metabolites.

FIGS. 5A-5B. (FIG. 5A) The effect of individual amino acids (2 mM) on HCT15 cell proliferation as measured by crystal violet assay at 48 hours in QMAP (n=5). (FIG. 5B) Tryptophan (2 mM) significantly decreased cell viability in four different cell lines (HT29, U2OS, 293T, 3T3-L1) in DMEM as measured by CellTiter-Glo assay at 24 hours (n=6). P-values were calculated by Student's t-test compared to control (A, QMAP B, Veh); * p<0.05,** p<0.01, *** p<0.001, **** p<0.0001.

FIGS. 6A-6B. (FIG. 6A) Experimental design for metabolic profiling. HCT116 cells were treated with tryptophan metabolites in QMAP media and analyzed via gas chromatography mass spectrometry (GC-MS). (FIG. 6B) Diagram of tryptophan (Trp) metabolism and metabolites tested in the experiment (blue).

FIGS. 7A-7F. (FIG. 7A) Heat map showing the Log2 fold change of TCA cycle intermediates after HCT116 cells have been treated with tryptophan metabolites for 6 hours (n=6) in QMAP media. (FIG. 7B) Relative fold changes of TCA cycle intermediates as measured by GC-MS profiling show that metabolites in the distal portion of the TCA cycle significantly decrease after 3-OH-Kyn treatment (n=3). (FIG. 7C) Schematic showing TCA cycle and pentose phosphate pathway (PPP) metabolites color-coded based on their direction of change following treatment with 3-OH-Kyn (2 mM) for 6 hours. (FIG. 7D) Relative fold changes of PPP intermediates as measured by GC-MS profiling indicate significant increases in PPP metabolite levels. (n=3). (FIG. 7E) Reduced (GSH) and oxidized glutathione (GSSG) show significant decreases with 3-OH-Kyn (2 mM), suggesting increased ROS (n=3-6). (FIG. 7F) Cell viability shows a dose-dependent decrease in three cell lines (293T, HCT116, U2OS) after 48 hours of 3-OH-Kyn treatment (n=3-6) in QMAP media. P-values were calculated by Student's t-test compared to untreated cells; * p<0.05, ** p<0.01, *** p<0.001, **** p<0.0001.

FIGS. 8A-8D. (FIG. 8A) Proposed schematic for increasing 3-OH-Kyn in cancer cells to mediate their death. (FIG. 8B) HCT116 IDO1 mRNA is increased with treatment of IFN-γ (50 ng/μL) for 24 hours. KYNU mRNA expression decreases after 48 hours of siRNA treatment (n=3-6). (FIG. 8C) Cell viability decreases after 24 and 48 hours of KYNU knockdown compared to non-targeting control. (FIG. 8D) DCFDA staining shows increased ROS with KYNU knockdown by flow cytometry. Annexin V and propidium iodide staining show increased apoptosis and cell death, respectively, after 48 hours of KYNU knockdown via flow cytometry (n=3-6). P-values were calculated by Student's t-test compared to control (CTRL) treatment or NT siRNA; ** p<0.01, *** p<0.001, **** p<0.0001.

FIGS. 9A-9B. (FIG. 9A) Xanthommatin is significantly increased when HCT116 cells are treated with 3HK (n=3-4) in QMAP media. (FIG. 9B) Xanthommatin is the only tryptophan metabolite to increase when HCT116 cells are treated with IFN-γ (50 ng/μL) for 24 hours and 1mM tryptophan for 6 hours (n=3-4) in QMAP media. P-values were calculated by Student's t-test compared to vehicle (veh) treatment; **** p<0.0001.

### DETAILED DESCRIPTION

### Cell Culture Media

The present disclosure relates to a Quantitatively Mapping Amino Acid Properties (QMAP) cell culture media where all amino acids are present at the same concentration. In certain embodiments, other ingredients are present to support cell growth and viability. Amino acids are a major class of nutrients that are investigated in hundreds (maybe thousands) of studies per year to understand their role in health and disease processes. Because cell culture studies provide a simplified environment where conditions can be tightly defined and controlled, cell culture models of amino acid function are critical to understanding the role of amino acids in biological systems. However, a current limitation of existing commercially available media is that they do not contain all amino acids and/or the amino acids that are included are present in a wide range of concentrations. These differences in amino acid concentrations confound experiments to systematically compare how changing the levels of individual amino acids in cell culture media regulate cellular metabolism and function. By defining a cell culture media that supports cellular growth and viability that has all amino acids at the same concentration, this invention enables systematic, quantitative investigations of how amino acids affect cellular function and metabolism that were not previously possible.

Because existing medias were developed based on the idea of adding different amounts of amino acids to replicate specific physiologic milieus or arrived at decades ago by their ability to support the growth of specific cells, the concept of adding the same concentration of all amino acids does not reside in the literature and is non-obvious. Conventional cell culture practice teaches away from adding uniform levels of amino acids because it is not physiologic. Existing medias aimed at replicating the in vivo cellular environment are likely overstated in their ability to do so. The present inventors, however, have discovered that rather than attempting to approximate physiology in a way that likely is not possible with cell culture approaches, medias that simplify experimental design and interpretation unexpectedly provided greater value in numerous contexts. As demonstrated herein throughout the scientific investigations, QMAP Cell Culture Media enhanced experiments designed to quantitatively compare the regulatory functions of individual amino acids, a positive feature that has innumerable applications in biomedical research. In one example (Example 3 below), the QMAP Cell Culture Media was used to investigate tryptophan metabolism.

### Amino Acids

In certain aspects, the QMAP Cell Culture Media comprises amino acids (an “amino acid component”), where the amino acids are present at a uniform concentration. As used herein, the term “uniform concentration” means that the concentration of the amino acid is within about # 10% of the concentration of the other amino acids. In certain aspects, the concentration of the amino acid is within about 5% of the concentration of the other amino acids.

In certain aspects, the QMAP Cell Culture Media comprises 18 amino acids In certain aspects, the amino acids are glycine, alanine, arginine, aspartic acid, cysteine, glutamic acid, histidine, isoleucine, leucine, lysine, methionine, phenylalanine, proline, serine, threonine, tryptophan, tyrosine, and valine.

In certain aspects, the QMAP Cell Culture Media comprises 19 amino acids In certain aspects, the amino acids are glycine, alanine, arginine, asparagine, aspartic acid, cysteine, glutamic acid, histidine, isoleucine, leucine, lysine, methionine, phenylalanine, proline, serine, threonine, tryptophan, tyrosine, and valine.

In certain aspects, the QMAP Cell Culture Media comprises 20 amino acids. In certain aspects, the amino acids are glycine, alanine, arginine, asparagine, aspartic acid, cysteine, glutamic acid, histidine, isoleucine, leucine, lysine, methionine, phenylalanine, proline, serine, threonine, tryptophan, tyrosine, valine and glutamine.

In certain aspects, the amino acids are natural (L-) amino acids. In certain aspects the amino acids are L-glycine, L-alanine, L-arginine, L-asparagine, L-aspartic acid, L-cysteine, L-glutamic acid, L-histidine, L-isoleucine, L-leucine, L-lysine, L-methionine, L-phenylalanine, L-proline, L-serine, L-threonine, L-tryptophan, L-tyrosine, L-valine, and L-glutamine.

In certain aspects, the amino acids are present at a final concentration of about 0.1 to 1 mM. (i.e., 0.1, 0.2, 0.3, 0.4, 0.5, 0.6, 0.7, 0.8, 0.9, or 1 mM) in the QMAP culture media. In certain aspects, the amino acids are present at a final concentration of about 0 2 to 06 mM media. In certain aspects, the amino acids are present at a final concentration of about 0.3 to 0.5 mM media. In certain aspects, the amino acids are present at a final concentration of about 0.3 mM media.

In certain aspects, an amino acid comprises a detectable label. In certain embodiments, the detectable label is a non-naturally abundant stable isotope. In certain aspects, the stable isotope is ¹³C. In certain aspects, the stable isotope is ¹⁵N, ²H, or ¹⁸O. In certain aspects, the detectable label is a radioisotope. In certain aspects, the radioisotope is ¹⁴C or ³H.

### Inorganic Salts

In certain aspects, the QMAP Cell Culture media comprises inorganic salts (an “inorganic salt component”). In certain aspects, the QMAP Cell Culture media comprises Sodium Bicarbonate (NaHCO₃). In certain aspects the QMAP Cell Culture media comprises Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O). Additional minerals and metals are added depending on the needs of the cells being cultured and what of them remained in the protein bound complement of dialyzed fetal bovine serum. These include in no specific order iron, zinc, copper, selenium, molybdenum, vanadium, manganese, chromium, cobalt, and possibly others, each in different possible formulations.

In certain aspects, the NaHCO3 is present at a final concentration of 10-45 mM, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 mM in the QMAP culture media. In certain aspects, the NaHCO₃ is present at a concentration of 24 mM in the QMAP culture media. In certain aspects the concentration of NaHCO₃ is determined based on incubator CO₂ concentration and the desired pH.

In certain aspects the inorganic salt component comprises Calcium Chloride (CaCl₂ anhydrous) is present at a final concentration of 0.3 to 3.0 mM, e.g., 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 mM [in the QMAP culture media. In certain aspects, the CaCl₂ anhydrous is present at a final concentration of 1.0 to 2.0 mM in the QMAP culture media. In certain aspects, the CaCl₂anhydrous is present at a concentration of 1.8018 mM in the QMAP culture media.

In certain aspects, the Magnesium Sulfate (MgSO₄ anhydrous) is present at a final concentration of 0.3 to 50 mM, e.g., 0.3, 0.4, 0.5, 0.6, 0.7, 0.8, 0.9, 1.0, 2, 3, 4, 5, 6, 7, 8, 9, 10, 15, 20, 25, 30, 35, 40, 45, or 50 mM in the QMAP culture media. In certain aspects, the MgSO₄anhydrous is present at a concentration of 1-10 mM in the QMAP culture media. In certain aspects, the MgSO₄ anhydrous is present at a concentration of 0.8139 mM in the QMAP culture media.

In certain aspects, the Potassium Chloride (KCl) is present at a final concentration of 2.0 to 20.0 mM, e.g., 2, 3, 4, 5, 6, 7, 8, 9, 10, 11, 12, 13, 14, 15, 16, 17, 18 19 or 20 mM in the QMAP culture media. In certain aspects, the KCl is present at a concentration of 4 to 7 mM in the QMAP culture media. In certain aspects, the KCl is present at a concentration of 5.3333 mM in the QMAP culture media.

In certain aspects, the Sodium Chloride (NaCl) is present at a final concentration of 60 to 160 mM, e.g., 60, 70, 80, 90, 100, 110, 120, 130, 140, 150, or 160 in the QMAP culture media. In certain aspects, the NaCl is present at a concentration of 90-130 mM in the QMAP culture media. In certain aspects, the NaCl is present at a concentration of 110.3448 mM in the QMAP culture media.

In certain aspects, the Sodium Phosphate monobasic (NaH₂PO₄—H₂O) is present at a final concentration of 0.3 to 3.0 mM, e.g., 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 mM in the QMAP culture media. In certain aspects, the NaH₂PO₄—H₂O is present at a concentration of 0.7 to 1.2 mM in the QMAP culture media. In certain aspects, the NaH₂PO₄—H₂O is present at a concentration of 0.9058 mM in the QMAP culture media.

### RPMI 1640 Vitamin Solation

In certain aspects, the QMAP Cell Culture media comprises a RPMI 1640 Vitamin Solution (an “RPMI 1640 vitamin solution”). In certain aspects, the RPMI 1640 Vitamin Solution comprises Biotin, Choline chloride, D-Calcium pantothenate, Folic Acid (folate), Niacinamide, Pyridoxine hydrochloride, Riboflavin, Thiamine hydrochloride, Vitamin B12, and/or i-Inositol. In certain aspects the RPMI 1640 vitamin solution further comprises Ammonium chloride and Para-aminobenzoic acid (PABA).

In certain aspects, the Biotin is present at a final concentration of 0.00003 to 0.1 mM, e.g., 0.00003, 0.00004, 0.00005, 0.00006, 0.00007, 0.00008, 0.00009, 0.00010, 0.00100, 0.00200, 0.00300, 0.00400, 0.00500, 0.00600, 0.00700, 0.00800, 0.00900, 0.001000, 0.00200, 0.00300, 0.00400, 0.00500, 0.00600, 0.00700, 0.00800, 0.00900, 0.01000, 0.02000, 0.03000, 0.04000, 0.05000, 0.06000, 0.07000, 0.08000, 0.09000, or 0.10000 mM in the QMAP culture media. In certain aspects, the Biotin is present at a concentration of 0.0006 to 0.0010 mM in the QMAP culture media. In certain aspects, the Biotin is present at a concentration of 0.0008 mM in the QMAP culture media.

In certain aspects, the Ammonium chloride is present at a final concentration of 0.00 to 0.1 mM, e.g., 0.01, 0.02, 0.03, 0.04, 0.05, 0.06, 0.07, 0.08, 0.09, 0.10 mMin the QMAP culture media. In certain aspects, the Ammonium chloride is present at a concentration of 0.03-0.05 mM in the QMAP culture media. In certain aspects, ammonium chloride is absent from the QMAP culture media, in the case of an investigation of ammonia metabolism. In certain aspects, the Ammonium chloride is present at a concentration of 0.0400 mM in the QMAP culture media.

In certain aspects, the Choline chloride is present at a final concentration of 0.005 to 0.1 mM, e.g., 0.005, 0.006, 0.007, 0.008, 0.009, 0.010, 0.011, 0.012, 0.013, 0.014, 0.015, 0.016, 0.017, 0.018, 0.019, 0.020, 0.021, 0.022, 0.023, 0.024, 0.025, 0.026, 0.027, 0.028, 0.029, 0.030, 0.04, 0.05, 0.06, 0.07, 0.08, 0.09, or 0.1 mM in the QMAP culture media. In certain aspects, the Choline chloride is present at a concentration of 0.015 to 0.030 mM in the QMAP culture media. In certain aspects, the Choline chloride is present at a concentration of 0.0214 mM in the QMAP culture media.

In certain aspects, the D-Calcium pantothenate is present at a final concentration of 0.0001 to 0.1 mM, e.g., 0.0001, 0.0002, 0.0003, 0.0004, 0.0005, 0.0006, 0.0007, 0.0008, 0.0009, 0.0010, 0.0100, 0.0200, 0.0000, 0.0400, 0.0500, 0.0600, 0.0700, 0.0800, 0.0900, 0.01000, 0.0200, 0.0300, 0.0400, 0.0500, 0.0600, 0.0700, 0.0800, 0.0900, or 0.1000 mM in the QMAP culture media. In certain aspects, the D-Calcium pantothenate is present at a concentration of 0.0003 to 0.0007 mM in the QMAP culture media. In certain aspects, the D-Calcium pantothenate is present at a concentration of 0.0005 mM in the QMAP culture media.

In certain aspects, the Folic Acid is present at a final concentration of 0.0005 to 0.1 mM, e.g., 0.0005, 0.0006, 0.0007, 0.0008, 0.0009, 0.0010, 0.0020, 0.0030, 0.0040, 0.0050, 0.0060, 0.0070, 0.00180, 0.0090, 0.0100, 0.0200, 0.0300, 0.0400, 0.0500, 0.0600, 0.0700, 0.0800, 0.0900, or 0.1000 mM in the QMAP culture media. In certain aspects, the Folic Acid is present at a concentration of 0.0015 to 0.0030 mM in the QMAP culture media. In certain aspects, the Folic Acid is present at a concentration of 0.0023 mM in the QMAP culture media.

In certain aspects, the Niacinamide is present at a final concentration of 0.001 to 0.1 mM, e.g., 0.001, 0.002, 0.003, 0.004, 0.005, 0.006, 0.007, 0.008, 0.009, 0.010, 0.020, 0.030, 0.040, 0.050, 0.060, 0.070, 0.080, 0.090, 0.0100or 0.100 mM in the QMAP culture media. In certain aspects, the Niacinamide is present at a concentration of 0.006 to 0.010 mM in the QMAP culture media. In certain aspects, the Niacinamide is present at a concentration of 0.0082 mM in the QMAP culture media.

In certain aspects, the Para-aminobenzoic acid (PABA) is present at a final concentration of, e.g., 0.005, 0.006, 0.007, 0.008, 0.009, 0.010, 0.011, 0.012, 0.013, 0.014, 0.015, 0.016, 0.0170.005 to 0.1 mM, 0.018, 0.019, 0.020, 0.021, 0.022, 0.023, 0.024, 0.025, 0.026, 0.027, 0.028, 0.029, 0.030, 0.04, 0.05, 0.06, 0.07, 0.08, 0.09, or 0.1 mM in the QMAP culture media. In certain aspects, PABA is absent from the QMAP culture media. In certain aspects, the PABA is present at a concentration of 0.005 to 0.010 mM in the QMAP culture media. In certain aspects, the Para-aminobenzoic acid is present at a concentration of 0.0073 mM in the QMAP culture media.

In certain aspects, the Pyridoxine hydrochloride is present at a final concentration of 0.001 to 0.1 mM, e.g., 0.001, 0.002, 0.003, 0.004, 0.005, 0.006, 0.007, 0.008, 0.009, 0.010, 0.020, 0.030, 0.040, 0.050, 0.060, 0.070, 0.080, 0.090, 0.0100or 0.100 mM in the QMAP culture media. In certain aspects, the Pyridoxine hydrochloride is present at a concentration of 0.030 to 0.070 mM in the QMAP culture media. In certain aspects, the Pyridoxine hydrochloride is present at a concentration of 0.001 to 0.1 mM 0.0049 mM in the QMAP culture media.

In certain aspects, the Riboflavin is present at a final concentration of 0.0001 to 0.1 mM, e.g., 0.0001, 0.0002, 0.0003, 0.0004, 0.0005, 0.0006, 0.0007, 0.0008, 0.0009, 0.0010, 0.0100, 0.0200, 0.0000, 0.0400, 0.0500, 0.0600, 0.0700, 0.0800, 0.0900, 0.01000, 0.0200, 0.0300,0.0400, 0.0500, 0.0600, 0.0700, 0.0800, 0.0900, or 0.1000 mM in the QMAP culture media. In certain aspects, the Riboflavin is present at a concentration of 0.0006 to 0.0010 mM in the QMAP culture media. In certain aspects, the Riboflavin is present at a concentration of 0.00085 mM in the QMAP culture media.

In certain aspects, the Thiamine hydrochloride is present at a final concentration of 0.0005 to 0.1 mM, e.g., 0.0005, 0.0006, 0.0007, 0.0008, 0.0009, 0.0010, 0.0020, 0.0030, 0.0040, 0.0050, 0.0060, 0.0070, 0.00180, 0.0090, 0.0100, 0.0200, 0.0300, 0.0400, 0.0500, 0.0600, 0.0700, 0.0800, 0.0900, or 0.1000 mM in the QMAP culture media. In certain aspects, the Thiamine hydrochloride is present at a concentration of 0.001 to 0.005 mM in the QMAP culture media. In certain aspects, the Thiamine hydrochloride is present at a concentration of 0.0030 mM in the QMAP culture media.

In certain aspects, Vitamin B12 is present at a final concentration of 0.000001 to 0.1 mM, e.g., 0.000001, 0.000002, 0.000003, 0.000004, 0.000005, 0.000006, 0.000007, 0.000008, 0.000009, 0.00001, 0.00002, 0.00003, 0.00004, 0.00005, 0.00006, 0.00007, 0.00008, 0.00009 0.00010, 0.00100, 0.00200, 0.00300, 0.00400, 0.00500, 0.00600, 0.00700, 0.00800, 0.00900, 0.001000, 0.00200, 0.00300, 0.00400, 0.00500, 0.00600, 0.00700, 0.00800, 0.00900, 0.01000 0.02000, 0.03000, 0.04000, 0.05000, 0.06000, 0.07000, 0.08000, 0.09000, or 0.10000 mM in the QMAP culture media. In certain aspects, the Vitamin B12 is present at a concentration of 0.000003 to 0.000005 mM in the QMAP culture media. In certain aspects, the Vitamin B12 is present at a concentration of 0.000004 mM in the QMAP culture media.

In certain aspects, the i-Inositol is present at a final concentration of 0.1 to 1.0 mM, e.g., 0.1, 0.2, 0.3, 0.4, 0.5, 0.6, 0.7, 0.8, 0.9 or 1.0 mM in the QMAP culture media. In certain aspects, the i-Inositol is present at a concentration of 0.1 to 0.3 mM in the QMAP culture media. In certain aspects, the i-Inositol is present at a concentration of 0.1944 mM in the QMAP culture media. In certain aspects i-Inositol is absent from the QMAP culture media.

### Auxiliary Ingredients

In certain aspects, the QMAP Cell Culture media comprises auxiliary ingredients (an “other ingredient component”), including D-Glucose, Sodium pyruvate, 3-Hydroxybutyric acid sodium salt, L-Carnitine hydrochloride, Sodium L-lactate, Penicillin-streptomycin, and/or Dialyzed fetal bovine serum.

In certain aspects, the auxiliary ingredient component in the QMAP Cell Culture Media comprises D-glucose, L-Carnitine hydrochloride and dialyzed fetal bovine serum.

In certain aspects, the D-Glucose is present at a final concentration of 0 to 45 mM, e.g., 5, 6, 7, 8, 9, 10, 11, 12, 13, 14, 15, 16, 17, 18, 19, 20, 21, 22, 23, 24, 25, 30, 35, 40 or 45 mM in the QMAP culture media. In certain aspects, the D-Glucose is present at a concentration of 5 to 25 mM in the QMAP culture media. In certain aspects, the D-Glucose is present at a concentration of 5 mM in the QMAP culture media. In certain aspects, D-Glucose is absent from the QMAP culture media.

In certain aspects, the Sodium pyruvate is present at a final concentration of 0 to 10 mM, e.g., 0.01, 0.02, 0.03, 0.04, 0.05, 0.06, 0.07, 0.08, 0.09. 0.1. 0.2, 0.3, 0.4, 0.5, 0.6, 0.7, 0.8, 0.9, 1.0.1.1, 1.2, 1.3, 1.4, 1.5, 1.6, 1.7, 1.8, 1.9, 2.0, 3.0, 4.0, 5.0, 6.0, 7.0, 8.0, 9.0, or 10 mM in the QMAP culture media. In certain aspects, the Sodium pyruvate is present at a concentration of 0.05 to 2.0 mM in the QMAP culture media. In certain aspects, the Sodium pyruvate is present at a concentration of 0.1 mM in the QMAP culture media. In certain aspects, Sodium pyruvate is absent from the QMAP culture media.

In certain aspects, the 3-Hydroxybutyric acid sodium salt is present at a final concentration of 0 to 10 mM, e.g., 0.01, 0.02, 0.03, 0.04, 0.05, 0.06, 0.07, 0.08, 0.09. 0.1. 0.2, 0.3, 0.4, 0.5, 0.6, 0.7, 0.8, 0.9, 1.0.1.1, 1.2, 1.3, 1.4, 1.5, 1.6, 1.7, 1.8, 1.9, 2.0, 3.0, 4.0, 5.0, 6.0, 7.0, 8.0, 9.0, or 10 in the QMAP culture media. In certain aspects, the 3-Hydroxybutyric acid sodium salt is present at a concentration of 0.01 to 5 mM in the QMAP culture media. In certain aspects, the 3-Hydroxybutyric acid sodium salt is present at a concentration of 0.05 mM in the QMAP culture media. In certain aspects, 3-Hydroxybutyric acid sodium salt is absent from the QMAP culture media.

In certain aspects, the L-Carnitine hydrochloride is present at a final concentration of 0 to 5.0 mM, e.g., 0.01, 0.02, 0.03, 0.04, 0.05, 0.06, 0.07, 0.08, 0.09. 0.1. 0.2, 0.3, 0.4, 0.5, 0.6, 0.7, 0.8, 0.9, 1.0.1.1, 1.2, 1.3, 1.4, 1.5, 1.6, 1.7, 1.8, 1.9, 2.0, 3.0, 4.0, 5.0 in the QMAP culture media. In certain aspects, the L-Carnitine hydrochloride is present at a concentration of 0.01 to 0.1 mM in the QMAP culture media. In certain aspects, the L-Carnitine hydrochloride is present at a concentration of 0.04 mM in the QMAP culture media. In certain aspects, L-Carnitine hydrochloride is absent from the QMAP culture media.

In certain aspects, the Sodium L-lactate is present at a final concentration of 0 to 20 mM, e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0, 6.0, 7.0, 8.0, 9.0, 10, 11, 12, 13, 14, 15, 16, 17, 18, 19 or 20 mM in the QMAP culture media. In certain aspects, Sodium L-lactate is present at a concentration of 0.1 to 5.0 mM in the QMAP culture media. In certain aspects, the Sodium L-lactate is present at a concentration of 0.9 mM in the QMAP culture media. In certain aspects, Sodium L-lactate is absent from the QMAP culture media.

In certain aspects, the Penicillin-streptomycin is present at a final concentration of 0 to 5×, e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0× in the QMAP culture media. In certain aspects, the Penicillin-streptomycin is present at a concentration of 0.5 to 2× in the QMAP culture media. In certain aspects, the Penicillin-streptomycin is present at a concentration of 1× in the QMAP culture media. In certain aspects, Penicillin-streptomycin is absent from the QMAP culture media.

In certain aspects, the Dialyzed fetal bovine serum is present at a final concentration of 0 to 20%, e.g., 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3.0, 4.0, 5.0, 6.0, 7.0, 8.0, 9.0, 10, 11, 12, 13, 14, 15, 16, 17, 18, 19, or 20% vol/vol in the QMAP culture media. In certain aspects, the Dialyzed fetal bovine serum is present at a concentration of 2% to 15% vol/vol in the QMAP culture media. In certain aspects, the Dialyzed fetal bovine serum is present at a concentration of 10% vol/vol in the QMAP culture media. In certain aspects, Dialyzed fetal bovine serum is absent from the QMAP culture media.

In certain aspects, the QMAP Cell Culture Media comprises D-glucose, L-Carnitine hydrochloride and dialyzed fetal bovine serum.

Additional minerals and metals are added depending on the needs of the cells being cultured and what of them remained in the protein bound complement of dialyzed fetal bovine serum. These include in no specific order iron, zinc, copper, selenium, molybdenum, vanadium, manganese, chromium, cobalt, and possibly others, each in different possible formulations.

### pH of QMAP Cell Culture Media

In certain aspects, the QMAP Cell Culture Media has a pH of about 7.4±0.5 (i.e., in the range of about pH 7.35 to about pH 7.45).

### Methods of Making QMAP Cell Culture Media

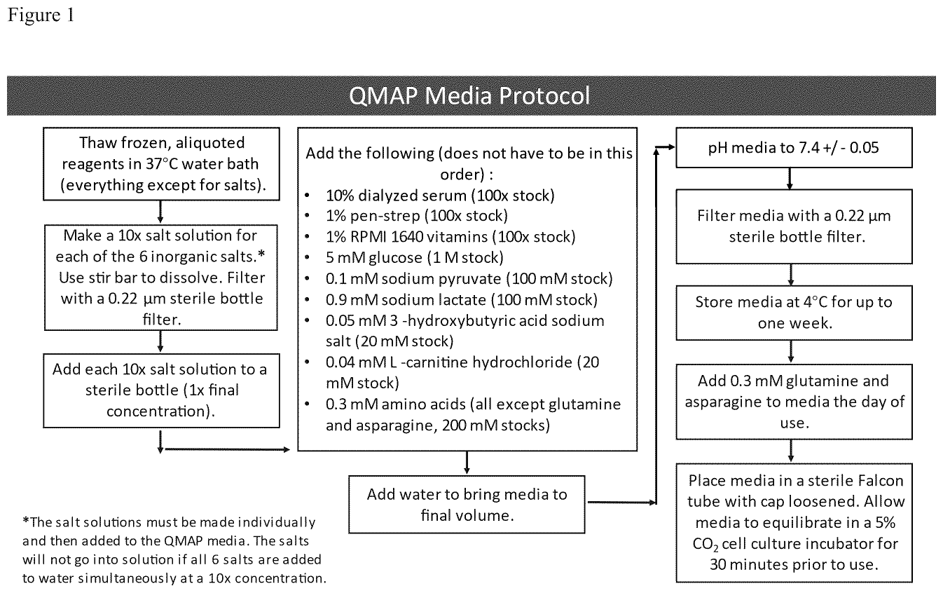

In certain aspects, a QMAP Cell Culture Media is prepared In one aspect, the QMAP Cell Culture Media is prepared as depicted in FIG. 1.

Briefly, individual 10× salt solutions are each made separately so that the salts will properly dissolve It is very important that the salt solutions are made individually and then added to the QMAP media. The salts will not go into solution if multiple salts are added to water simultaneously at a 10× concentration. The individual salt solutions are filtered using a 0.22 um sterile bottle filter.

Each 10× salt solution is added to a sterile bottle (1× final concentration). In certain aspects, Sodium Bicarbonate (NaHCO₃) is included. Additionally, Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O) is added.

In one aspect, the following ingredients are added to the solution (does not have to be in this order):

dialyzed serum (e.g., 10% dialyzed serum (100% stock))

optional antibiotic (e.g., 1% pen-strep (100× stock))

RPMI 1640 vitamins (e.g., 1% RPMI 1640 vitamins (100× stock))

glucose (e.g., 5 mM glucose (1 M stock))

optional sodium pyruvate (e.g., 0.1 mM sodium pyruvate (100 mM stock))

optional sodium lactate (e.g., 0.9 mM sodium lactate (100 mM stock))

optional 0.05 mM 3-hydroxybutyric acid sodium salt (20 mM stock))

L-carnitine hydrochloride (e.g., 0.04 mM L-carnitine hydrochloride (20 mM stock))

amino acids (all except glutamine and asparagine) (e.g., 0.3 mM amino acids (all except glutamine and asparagine), 200 mM stocks))

supplemental minerals and metals.

Next, water is added to the solution to bring the media up to the final volume.

The pH is adjusted to be at a pH of 7.4±0.05.

In certain aspects, dialyzed serum is added after pH adjusting.

Finally, the media is sterilized, such as with a 0.22 μm sterile bottle filter

The media can be stored at 4° C. for up to one week. The QMAP media lacking asparagine and glutamine can be stored at 4° C. for several weeks.

Just prior to use of the QMAP Media, glutamine and asparagine (e.g., 0.3 mM, if that is the concentration of the other amino acids) to media the day of use. The media is then placed in a sterile Falcon tube with cap loosened. The media is allowed to equilibrate in a 5% CO₂ cell culture incubator for 30 minutes prior to use.

In one aspect, following ingredients are added to the solution (does not have to be in this order):

(a) adding to a sterile container
(i) RPMI 1640 vitamin solution,
(ii) glucose,
(iii) L-carnitine,
(iv) an amino acid solution consisting of glycine, alanine, arginine, aspartic acid, cysteine, glutamic acid, histidine, isoleucine, leucine, lysine, methionine, phenylalanine, proline, serine, threonine, tryptophan, tyrosine, valine, wherein the amino acid solution comprises a uniform concentration of all the amino acids;
(v) glutamine solution, wherein the glutamine is present at the same concentration as the amino acids in (v),
(vi) asparagine solution, wherein the asparagine is present at the same concentration as the amino acids in (v);
(vii) optionally adding antibiotic, sodium pyruvate, sodium lactate, 3-Hydroxybutyric acid sodium salt, and/or minerals and metals,

(b) adding water to bring media to predetermined volume to form an intermediate media;

(c) adjusting the pH of the intermediate media to about 7.4±0.5; and

(d) adding a salt solution consisting of 10× Sodium Bicarbonate (NaHCO₃) to a sterile container to form a secondary media;

(e) sterilizing the secondary media through 0.22 μm filter;

(f) adding dialyzed fetal bovine serum to form the QMAP cell culture media, and

(g) exposing the media to 5% CO₂ in a sterile environment or cell culture incubator.

### QMAP Cell Culture Media Kits

In certain aspects, kits comprise a first container comprising QMAP Cell Culture Media lacking glutamine and/or asparagine, a second container comprising glutamine and/or asparagine, and instructions for preparing the final, complete QMAP Cell Culture Media that contains all 20 amino acids.

### Methods of Use

In certain aspects, cells are cultured in small scale cultures using the QMAP Cell Culture Media. In certain aspect the cells are cultured in 125 ml containers having about 25 mL of media, 250 ml containers having about 50 to 100 mL of media, 500 mL containers having about 100 to 200 mL of media. Alternatively, the cultures can be large scale such as for example 1000 mL containers having about 300 to 1000 mL of media, 3000 mL containers having about 500 mL to 3000 mL of media. 8000 mL containers having about 2000 mL to 8000 mL of media, and 15000 mL containers having about 4000 mL to 15,000 mL of media. Cultures for manufacturing can contain 10,000 L of media or more. Concentrated media may be based on any cell culture media formulation. Such a concentrated media can contain most of the components of the cell culture media at, for example, about 5×, 6×, 7×, 8×, 9×, 10×, 12×, 14×, 16×, 20×, 30×, 50×, 100×, 200×, 400×, 600×, 800×, or even about 1000× of their normal useful amount. Concentrated media are often used in batch culture processes.

In certain aspects, the cells are mammalian cells, avian cells, insect cells, and yeast cells.

The invention will now be illustrated by the following non-limiting Examples.

### Example 1

### Quantitatively Mapping Amino Acid Properties (QMAP) Cell Culture Media

Quantitatively Mapping Amino Acid Properties (QMAP) Cell Culture Media is a chemically-defined cell culture media with all amino acids at the same concentration. In the present Example, all amino acids were present in the QMAP Cell Culture Media at a concentration of 0.3 mM. This media was designed for understanding the effects of amino acids on metabolism, metabolomics, and other aspects of cellular function in cell culture studies.

Ingredients were bought or made in bulk and frozen in aliquots at −20° C. One day prior to cell culture, reagents were thawed in a 37° C. water bath and combined. pH was adjusted to 7.4 +/−0.05, and media was filtered using a 0.22 μm sterile bottle-top filter. Media was kept at 4° C. until time of use. Due to its sensitivity to degradation, glutamine was not added to media until time of use.

| QMAP Cell Culture Media | (mM) |
|---|---|
| Amino Acids | |
| L-glycine | 0.3 |
| L-alanine | 0.3 |
| L-arginine | 0.3 |
| L-asparagine | 0.3 |
| L-aspartic acid | 0.3 |
| L-cysteine | 0.3 |
| L-glutamic acid | 0.3 |
| L-glutamine | 0.3 |
| L-histidine | 0.3 |
| L-isoleucine | 0.3 |
| L-leucine | 0.3 |
| L-lysine | 0.3 |
| L-methionine | 0.3 |
| L-phenylalanine | 0.3 |
| L-proline | 0.3 |
| L-serine | 0.3 |
| L-threonine | 0.3 |
| L-tryptophan | 0.3 |
| L-tyrosine | 0.3 |
| L-valine | 0.3 |

| Inorganic Salts | mM |
|---|---|
| Calcium Chloride (CaCl2 anhyd.) | 1.8018 |
| Magnesium Sulfate (MgSO4 anhyd.) | 0.8139 |
| Potassium Chloride (KCl) | 5.3333 |
| Sodium Bicarbonate (NaHCO3) | 24 |
| Sodium Chloride (NaCl) | 110.3448 |
| Sodium Phosphate monobasic (NaH2PO₄—H₂O) | 0.9058 |

| RPMI 1640 Vitamin Solution 100x | mM |
|---|---|
| Biotin | 0.0008 |
| Ammonium chloride | 0.0400 |
| Choline chloride | 0.0214 |
| D-Calcium pantothenate | 0.0005 |
| Folic Acid | 0.0023 |
| Niacinamide | 0.0082 |
| Para-aminobenzoic acid | 0.0073 |
| Pyridoxine hydrochloride | 0.0049 |
| Riboflavin | 0.0005 |
| Thiamine hydrochloride | 0.0030 |
| Vitamin B12 | 0.000004 |
| i-Inositol | 0.1944 |

| Other | mM |
|---|---|
| D-Glucose | 5 |
| Sodium pyruvate | 0.1 |
| 3-Hydroxybutyric acid sodium salt | 0.05 |
| L-Carnitine hydrochloride | 0.04 |
| Sodium L-lactate | 0.9 |
| Penicillin-streptomycin (10,000 U/mL) | 1x |
| Dialyzed fetal bovine serum | 10x |

### Example 2

### A Systematic Cell Culture Screen Of Individual Amino Acids Reveals Substrate- And Signaling-Based Regulation Of Metabolism

### ABSTRACT

Although amino acids are often referred to as the building blocks of proteins, they are also an important class of bioactive metabolites that regulate metabolism through their roles as reaction substrates (material) and signaling (information). As material, amino acids contribute carbon or nitrogen that is essential for synthesis of fatty acids, nucleotides/bases, signaling molecules, and epigenetic modifiers. As information, amino acids and their metabolites directly alter enzymatic activities through allostery or indirectly through modulation of enzymatic regulatory proteins.

Although amino acid regulation has been studied for decades, new discoveries are still being made, indicating that many gaps in common understanding remain. To map the global organization of amino acid metabolism in cell culture, a systematic activity metabolomics assay consisting of two screens was performed. The goal of the first screen was to determine if an amino acid provides material for metabolite synthesis. To accomplish this goal, human primary dermal fibroblasts (HDFn), human embryonic kidney cells (293T), and human colon cancer cells (HCT116) were treated with either heavy (¹³C) carbon-labeled or heavy (¹⁵N) nitrogen-labeled amino acids and performed stable isotope tracing to detect ¹³C or ¹⁵N enrichment into ˜200 metabolites. If a metabolite in cells treated with a ¹³C amino acid was labeled with ¹³C, then the amino acid had supplied material for that metabolite.

### Introduction

Metabolism encompasses all chemical reactions within a cell and is thus a fundamental property of living systems. Metabolomics, the study of small molecules or metabolites, historically has been limited to detection of metabolites as toxicology indicators or disease biomarkers via NMR or mass spectrometry. The notion that metabolomics technology could be used to screen for bioactive metabolites that drive metabolic processes and modulate phenotypes is an emerging concept, aptly named “activity metabolomics.” Activity metabolomics studies often utilize high-throughput, in vitro screens that are designed to systematically measure global changes in the metabolome or proteome that are associated with bioactive metabolites. Many studies have focused on the discovery of novel protein-metabolite interactions, while others have identified metabolites that alter physiological or pathological cell phenotypes. The practical implication of this knowledge is that bioactive metabolites or their synthetic analogues could be used to metabolically alter cell state and disease.

Surprisingly, activity metabolomics has yet to be applied broadly to amino acids, an important class of metabolites that regulates metabolism through their roles as material and signaling information. As material, amino acids contribute carbon or nitrogen that is essential for synthesis of fatty acids, nucleotides/bases, signaling molecules, and epigenetic modifiers. As signaling information, amino acids directly alter enzymatic activities through allostery or indirectly through modulation of enzymatic regulatory proteins. Though the regulatory functions of amino acids have been studied for decades, how each amino acid distinctively regulates the cellular metabolic network remains poorly understood.

To address this issue, we devised an original activity metabolomics assay utilizing side-by-side treatment of cells with individual amino acids. Broad questions were sought to be addressed, such as, which components of the metabolome are more flexible or rigid in response to amino acids? What is the metabolome-wide contribution of an amino acid's carbon or nitrogen? Are non-essential amino acids used differently than essential amino acids? More specific questions relevant to disease were also sought to be addressed. For example, type 2 diabetes is associated with high levels of circulating branch-chain amino acids (BCAAs). Thus, how do BCAAs alter cellular metabolism when given to cells in high doses? By isolating the metabolic regulatory activities of individual amino acids via our activity metabolomics assay, new ways that amino acids regulate metabolism as material and signaling information were also discovered. The present screen also indicates a novel regulatory function for tryptophan or its products as an inhibitor of glutamine metabolism. Overall, this study shows how an activity metabolomics assay was applied to amino acids in cell culture to reveal novel ways that amino acids regulate cellular metabolism through carbon/nitrogen contribution or metabolic enzyme regulation.

### Method

### Cell Culture

HCT-116, HEK293T, and the primary human dermal fibroblast, neonatal (HDFn), were purchased from ATCC and were under passage 10 for all the experiments. HCT-116 and HEK293T were propagated in DMEM with 10% FBS (R&D Systems, Cat #S11550), 1% penicillin/streptomycin (Gibco, Cat #15140122) and 1% Glutamax (Gibco, Cat #35050061). For HDFn, the cells were propagated in fibroblast basal complete, which is comprised of fibroblast basal medium (ATCC, Cat #PCS-201-030), fibroblast growth kit (ATCC, Cat #PCS-201-040) and 10% FBS.

### Quantitatively Mapping Amino Acid Properties (QMAP) Cell Culture Media

Ingredients were bought or made in bulk and frozen in aliquots at −20° C. One day prior to cell culture, reagents were thawed in a 37° C. water bath and combined. pH was adjusted to 7.4 +/−0.05, and media was filtered using a sterile bottle-top filter of 0.22 μm. Media were kept at 4° C. until time of use. Due to its sensitivity to degradation, glutamine was not added to media until time of use. The QMAP for the HDFn cells was also supplemented with a fibroblast growth kit.

### Seeding and Dosing Cells

Cells were counted and plated on poly-D-lysine (Sigma, #P6407) coated 6-well plates (Avantor, #10861-696) at 4.5×10⁵/well for HCT-116 and HDFn and 7.5×10⁵/well for HEK293T. For the plating, HCT-116 and HEK293T cells were grown in 50% DMEM/QMAP overnight while the HDFn cells were grown in 50% fibroblast basal complete/QMAP+fibroblast growth kit. Media was changed to QMAP plus individual amino acid treatments at the start of the experiment. 200 mM amino acid stocks were used to make the QMAP+individual amino acid treatment medias that were placed in the tissue culture incubator for 30-45 minutes prior to the start of the dosing for equilibration of media bicarbonate with incubator CO2. For metabolomic profiling experiments, cells were treated with additional amino acid doses of 0.5, 1 and 2 mM, and 1 mM was used for stable isotope tracing experiments. To minimize systematic error, each amino acid dose was added as a batch (20 amino acids+vehicle control) such that each well of each plate contained a different amino acid. The process was repeated for the other two concentrations of amino acids on the next plates to make set. This was repeated four more times to achieve the sample size of 5 replicates for each of the 3 doses of amino acids. Each plate was then incubated for 6 hours in the tissue culture incubator before being rapidly washed twice with sterile water and snap frozen on liquid nitrogen before being stored at −80° C. The next day, the plates were lyophilized for 90 minutes and then stored at −80° C.

### Metabolite Extraction

Extraction buffer was prepared by adding 2:2:1 methanol: acetonitrile: water. For profiling, the following internal standards (Cambridge Isotope Laboratories) were added at 1 μg/mL each: L-Valine-D8, L-Aspartate (¹³C4, D3, ¹⁵N) and Succinate (13C4D4). 6-well plates were removed from storage at −80° C. and 1 ml of ice-cold extraction buffer was added. Plates were incubated at −20° C. for 5 minutes, scraped, transferred to microcentrifuge tubes and frozen in liquid nitrogen. Tubes were then placed into a sonicating water bath for 10 minutes and rotated at −20° C. for 1 hour before being centrifuged at 4° C. for 10 minutes at 21,000×g. 300 ml of extract was dried down completely using a speed-vacuum (˜90 min), dissolved in 30 ml of acetonitrile: water (1:1), vortexed for 10 min, and stored overnight at −20° C. to allow protein precipitation. Samples were then centrifuged at 4° C. for 10 minutes at 21,000×g and the supernatants were transferred into autosampler vials for analysis. For the QCs, 50-75 ml of each extract was pooled, at the step prior to drying down, mixed and dried in 300 ml amounts alongside the samples. Once dried, QCs were processed with the samples but recombined prior to being aliquoted into autosampler vials. All samples were then frozen at −80° C., and only one set of samples, which included a QC aliquot, was thawed, vortexed and placed into the instrument. This resulted in the samples only remaining at 4° C. for 12-15 hours and allowed the inclusion of a QC that was thawed at the same time as the samples.

### LC-MS Method and Data Analysis

2 ml of each sample were separated on Millipore SeQuant ZIC-PHILIC (2.1×150 mm, 5 μm particle size) with a ZIC-PHILIC guard column (20×2.1 mm) using a Thermo Q Exactive hybrid quadrupole Orbitrap mass spectrometer with a Vanquish Flex UHPLC system or Vanquish Horizon UHPLC system.

The mobile phase was run at a flow rate of 0.150 mL/minute and contained Buffer A [20 mM (NH₄)₂CO₃, 0.1% NH₄OH (v/v)] and Buffer B [acetonitrile] with the following linear gradients: 0-20 minutes Buffer A from 20 to 80%, 20-20.5 minutes Buffer A from 80 to 20% and 20.5-28 minutes held at 20% Buffer A. The mass spectrometer was operated in full-scan, polarity-switching mode from 1 to 20 minutes, with the spray voltage set to 3.0 kV, the heated capillary held at 275° C. and the HESI probe held at 350° C. The sheath gas flow was set to 40 units, the auxiliary gas flow was set to 15 units and the sweep gas flow was set to 1 unit. For the profiling, MS data acquisition was performed in a range of m/z 70-1,000, with the resolution set at 70,000, the AGC target at 1×106 and the maximum injection time at 200 ms (PMID: 28388410). For tracing, 2 or 3 ml of sample was injected twice in either positive or negative mode to maximize detection efficiency and minimize metabolite crowding using the Sim-mode.

LC-MS data was processed using the Thermo Scientific TraceFinder 5.1 software. Targeted metabolites were identified based on the University of Iowa Metabolomics Core facility standard-confirmed, in-house library defining a target ion and at least 1 confirming ion and accurate mass, retention time, and MS/MS fragmentation pattern when present.

### RESULTS

### Activity Metabolomics Screen Design

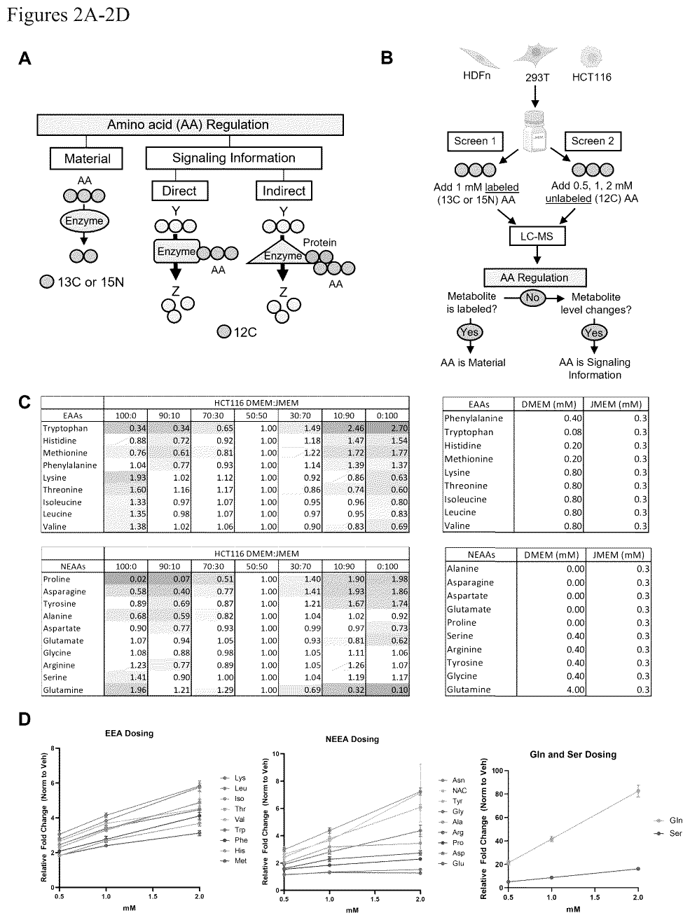

An activity metabolomics screen was designed to test how all 20 amino acids regulate cellular metabolic networks through their roles as material (substrate) and information (non-direct flux/signaling) (FIGS. 2A-2B). Three cell lines were incubated in QMAP and treated with either heavy (¹³C) carbon-labeled or heavy (¹⁵N) nitrogen-labeled amino acids, and stable isotope tracing was performed to detect ¹³C or ¹⁵N enrichment into ˜200 metabolites (Screen 1).

If a metabolite in cells treated with a ¹³C amino acid was labeled with ¹³C, then the amino acid had supplied material to produce that metabolite. The three cell lines were then treated with 0.5, 1, and 2 mM of individual, unlabeled amino acids, and metabolic profiling was performed to determine how individual amino acids quantitatively altered metabolite abundance (Screen 2). If a metabolite showed no ¹³C labeling from a ¹³C amino acid from the first screen but the relative abundance of that metabolite changed with the same unlabeled amino acid treatment, then the amino acid had supplied information to alter the metabolite level.

### Activity Metabolomics Screen Requirements and Rationale

Media formulation: Because commercially available cell culture media such as DMEM or RPMI contain variable concentrations of amino acids, a chemically defined, custom media was engineered with all amino acids present at 0.3 mM (QMAP). We chose 0.3 mM because the average amino acid concentration (excluding glutamine) is 0.477 mM for DMEM and 0.237 mM for RPMI.

Cell lines: Three human cell lines that were predicted to be metabolically divergent from one another were selected: primary fibroblasts (HDFn), embryonic kidney cells (293T), and colon cancer cells (HCT116). The rationale was that performing the assay in three, metabolically diverse cell lines would allow the identification of areas of metabolic rigidity and areas of metabolic flexibility from the screens, and whether these properties were conserved across cells lines.

Amino acid scalability in commercial media: To ensure that the linear scaling of amino acids was not an artifact of culturing in QMAP, a media dilution experiment was performed. All three cell lines were treated with the following ratios of DMEM to QMAP: 100:0, 90:10, 70:30, 50:50, 30:70, 10:90, and 0:100. The intracellular fold change of each amino acid compared with untreated cells via LC-MS was then measured. Amino acids at high concentrations in DMEM compared to QMAP decreased in intracellular concentration with each media dilution. Conversely, amino acids that were absent or present at low concentrations in DMEM increased linearly with increasing amounts of QMAP (FIG. 2B).

Amino acid dosing: To ensure that the amino acid dosing corresponded with near linear increases in amino acid uptake into cells, all cell lines were cultured in QMAP, the custom media, and treated with 0.5, 1, and 2 mM of each amino acid. the intracellular fold change was then measured of each amino acid compared with untreated cells via LC-MS (FIG. 2C). Most amino acids scaled near linearly at these doses, which informed the selection of the 1 mM dose for our ¹³C and ¹⁵N tracing studies.

Batch Effects: Given the magnitude of our screen design, batch effects were anticipated. For example, Screen 2 alone yielded 945 samples (3 cell lines×20 amino acids+1 vehicle×3concentrations×n=5) and 21.6 days of run time on the mass spectrometer. To minimize batch effects in experimental design, all reagents used in the custom-made media (QMAP) were ordered or made in bulk, aliquoted, and frozen for single use at −20° C. One cell line was used at a time, and the same two experimentalists seeded, treated, and harvested the cells in the same manner. Each well in the 6-well cell culture plates contained a different amino acid at the same dose, so that technical replicates for treatments were not allocated to the same plate or run sequentially on the instrument. See methods for LC-MS sample processing.

Screen 1: Stable ¹³C and ¹⁵N isotope tracing reveals known and unknown material contributions of amino acids

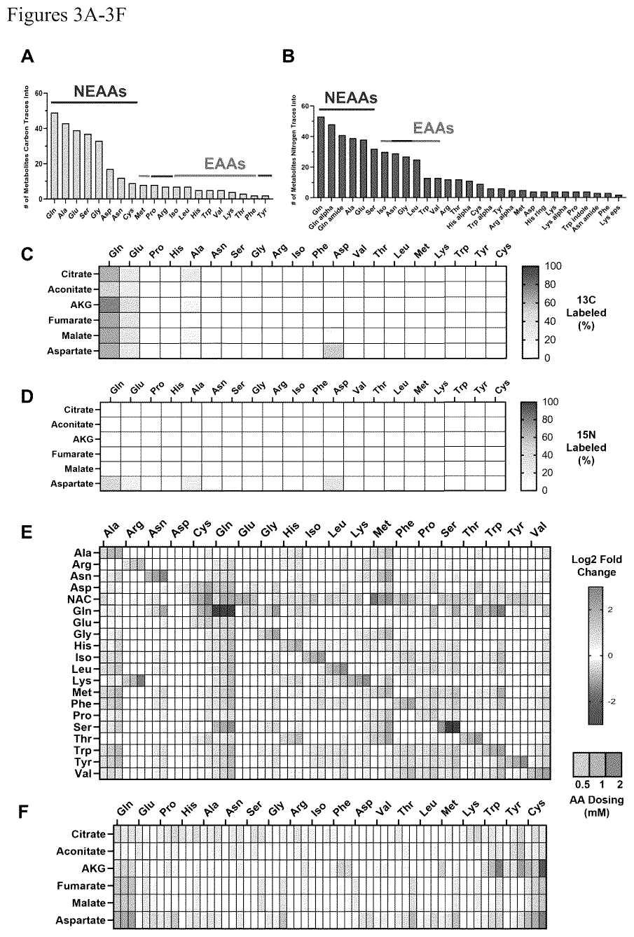

To compare carbon and nitrogen use from individual amino acids following ¹³C and ¹⁵N tracing, respectively, amino acids were ranked based on how many metabolites they supplied labeled carbon or nitrogen to. This resulted in a striking bifurcation of non-essential amino acids (NEAAs) and essential amino acids (EAAs), with NEAAs comprising the top 8 carbon-supplying amino acids (FIG. 3A) and the top 6 nitrogen-supplying amino acids (FIG. 3B). Glutamine and alanine contributed carbon (FIG. 3A) and nitrogen (FIG. 3B) to more metabolites than any other amino acid, illustrating their metabolic lability. Accordingly, glutamine and alanine are also the amino acids with the highest concentrations in human plasma and cell culture media such as DMEM and RPMI.

Stable isotope tracing also revealed known and unknown ways in which specific metabolites use carbon from amino acids. Glutamine, glutamate, aspartate, and alanine are known to supply carbon and nitrogen to the TCA cycle, which was observed when cells were treated with those labeled amino acids (FIGS. 3C-3D). Asparagine also contributed ¹³C to a small percentage of labeled aspartate (8%), citrate (3%), fumarate (5%), and malate (5%), suggesting conversion of asparagine to aspartate and then uptake of aspartate into the TCA cycle. This is consistent with literature showing that asparagine can provide carbon to sustain TCA cycle flux under low glutamine conditions.

### Screen 2: Metabolic Profiling Reveals Known and Unknown Examples of Amino Acids as Signaling Information

When cells were treated with 0.5, 1, and 2 mM of individual, unlabeled amino acids, the intracellular amount of the amino acid used for treatment increased in a dose-dependent manner relative to untreated cells (FIG. 3E); however, some amino acids exhibited differences in cellular accumulation that appeared to be cell line-specific. For example, the relative abundance of glutamine increased by ˜80 fold for the 2 mM dose in HCT116 cells compared to glutamate, which increased by 1.25-fold for the same dose. This phenomenon has been observed in previous amino acid dosing experiments with HCT116 cells, but not 293Ts or fibroblasts, suggesting that there are key differences in plasma membrane transporter expression, intracellular metabolism, and/or activity for certain amino acids in HCT116 cells. Interestingly, treatment of glycine led to a proportional, dose-dependent increase in serine in HCT116 cells;

but, conversely, treating cells with serine did not increase glycine levels. Though the enzyme converting serine to glycine is reversible, the only known way to synthesize glycine is from serine, suggesting that HCT116 cells may prefer to utilize serine over glycine.

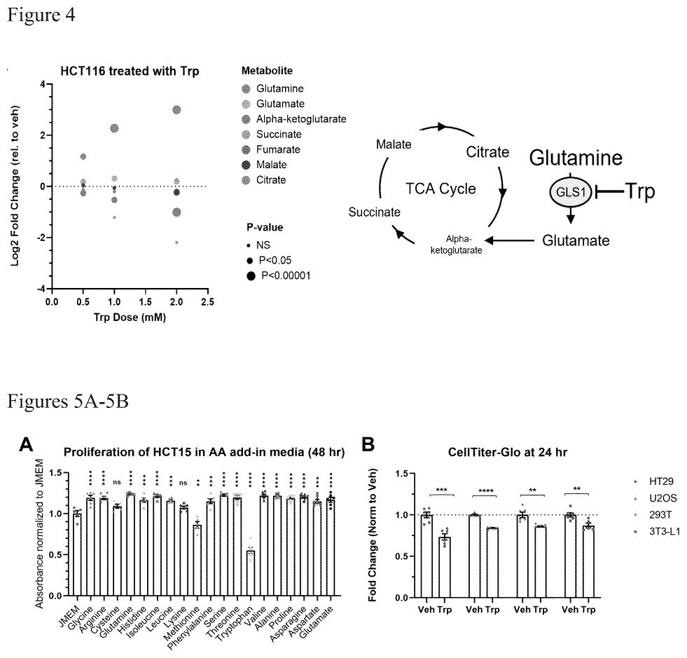

Metabolic profiling also revealed known and unknown ways in which metabolite levels were altered with amino acid treatments. Predictably, TCA cycle intermediates increased with glutamine and glutamate treatment (FIG. 3F), indicating glutamine/glutamate anaplerosis and metabolic regulation by provision of material, which was confirmed by ¹³C tracing in Screen 1. Surprisingly, unlabeled tryptophan decreased distal TCA cycle metabolite levels (alpha-ketoglutarate to malate) and increased glutamine levels in a dose-dependent manner despite no evidence of ¹³C labeling of glutamine or TCA cycle metabolites from ¹³C tryptophan (FIG. 4A). These results suggest that tryptophan can act as signaling information rather than material to disrupt glutamine anaplerosis via direct or indirect inhibition of glutaminase activity (FIG. 4A schematic).

### Discussion

Using a systematic, in vitro activity metabolomics assay, we investigated how amino acids regulate metabolism through their dual roles as material and signaling information. This was accomplished through the implementation of two metabolomics screens and an analysis of adjacent metabolite ratios within metabolic pathways. The first screen was designed to filter which amino acids function as material for the metabolome. Amino acids function as material when they contribute a carbon, nitrogen, or both, to a metabolite. Accordingly, individual, universally labeled ¹³C and ¹⁵N amino acids were added to cell culture media and traced into ˜200 metabolites. Stable isotope tracing revealed that the non-essential amino acids (NEAAs) traced more widely into the metabolome than essential amino acids (EEAs), suggesting tighter regulation of EAA pools. Tracing of amino acids into many known pathways was observed, such as glutamine, alanine, aspartate, and asparagine carbon into TCA cycle metabolites.

The second screen was designed to measure relative changes in metabolites when cells were treated with increasing doses of individual, unlabeled amino acids. As previously observed, amino acids such as glutamine and glutamate increased TCA cycle metabolites and aspartate. Because the first screen confirmed that glutamine and glutamate supplied carbon to the TCA cycle, the increases in TCA metabolite levels were attributed to glutamine and glutamate acting at least in part as material. The second screen also showed dose-dependent increases in glutamine and dose-dependent decreases in the distal TCA cycle metabolite levels when cells were treated with tryptophan. Because the first screen showed no evidence for ¹³C or ¹⁵N tracing into glutamine or any TCA cycle intermediates from labeled tryptophan, tryptophan is likely acting as signaling information to inhibit glutamine flux into the TCA cycle. Because many cancers rely on increased glutamine metabolism and anaplerosis, a tryptophan analogue that inhibits glutamine metabolism may show promise as a cancer therapeutic. Overall, this study demonstrated how activity metabolomics was applied to amino acids to discover new ways that amino acids regulate cellular metabolism by acting as material or signaling information.

### Example 3

### 3-Hydroxykynurenine Is a Cytotoxic Tryptophan Metabolite That Produces Xanthommatin in Human Cells

### Abstract

Tryptophan is an essential amino acid that is extensively characterized as a regulator of cellular function through its metabolism by indoleamine 2,3-deoxygenase (IDO) into the kynurenine pathway. However, despite decades of research on tryptophan metabolism, the metabolic regulatory roles of it and many of its metabolites are not well understood. To address this, an activity metabolomics screen of tryptophan and most of its known metabolites in cell culture was performed. It was discovered that treatment of human colon cancer cells (HCT116) with 3-hydroxykynurenine (3-HK), a metabolite of kynurenine, potently disrupted TCA cycle function. Citrate and aconitate levels were increased, while isocitrate and all downstream TCA metabolites were decreased, suggesting decreased aconitase function. It was hypothesized that 3HK or one of its metabolites increased reactive oxygen species (ROS) and inhibited aconitase activity. Accordingly, almost complete depletion of reduced glutathione and a decrease in total glutathione levels were observed. A dose-dependent decrease in cell viability after 48 hours of 3HK treatment was observed. These data suggest that raising the intracellular levels of 3HK could be sufficient to induce ROS-mediated apoptosis. The intracellular levels of 3HK were modulated by combined induction of IDO and knockdown of kynureninase (KYNU) in HCT116 cells. Cell viability decreased significantly after 48 hours of KYNU knockdown compared to controls, which was accompanied by increased ROS production and Annexin V staining revealing apoptosis. Finally, xanthommatin production was identified from 3-HK as a candidate radical-producing, cytotoxic mechanism. This work indicates that KYNU may be a target for disrupting tryptophan metabolism. It is interesting to note that many cancers exhibit overexpression of IDO, providing a cancer-specific metabolic vulnerability that could be exploited by KYNU inhibition.

### Introduction

Tryptophan is an essential amino acid that is used for protein synthesis, neurotransmitter production, and de novo NAD+ synthesis via the kynurenine pathway. Almost 95% of available tryptophan is directed through the kynurenine pathway into metabolites that can exhibit immunosuppressive properties, neuroprotective properties, or neurotoxic effects. In cancer, kynurenine has a potent immunosuppressive effect on the tumor microenvironment. Tumor overexpression of indoleamine 2,3-deoxygenase (IDO), the rate-limiting enzyme of the kynurenine pathway, is associated with increased levels of kynurenine, which can bind to the aryl hydrocarbon receptor and decrease T cell effector function and proliferation. In neurodegenerative diseases, kynurenine pathway metabolites that are downstream of kynurenine have been shown to have either a neuroprotective effect (kynurenic acid) or neurotoxic effect (quinolinic acid). Quinolinic acid can stimulate N-methyl-D-aspartate (NMDA) receptors leading to excitatory neuron death, while kynurenic acid acts as an antagonist for ionotropic glutamate receptors, and thus protects against excitotoxicity. Other kynurenine pathway metabolites, such as 3-hydroxykynurenine (3HK) have also been implicated in neuronal toxicity. At low levels, 3HK functions as an antioxidant and can scavenge reactive oxygen species (ROS) such as peroxyl radicals. At high levels or under oxidative conditions, 3HK can produce ROS at concentrations that induce apoptosis and neuronal death. Increased levels of 3HK in the brain have been associated with several neurodegenerative diseases, including Huntington's Disease (HD). It is interesting to note that in a transgenic mouse model of HD, 3HK was the only kynurenine pathway metabolite to increase following an intrastriatal injection of kynurenine. Furthermore, brain tissue from these mice exhibited significantly lower activity of kynureninase, the enzyme responsible for metabolizing 3HK in mammals.

Overall, the tryptophan metabolite literature can be summarized by binning metabolites into two main disease contexts: 1) immunosuppression that may contribute to tumor progression (e.g., kynurenine), and 2) neurotrophic or neurotoxic (e.g., kynurenic acid, quinolinic acid, 3-hydroxykynurenine). However, it is unclear whether tryptophan metabolites that cause neuronal toxicity could also exert a cytotoxic effect on cancer cells. Given that 3HK is thought to induce neuronal apoptosis via ROS and not through excitotoxicity mechanisms, 3HK could potentially exert cytotoxic effects against cancer cells, especially in IDO-overexpressing tumors that exhibit high tryptophan consumption.

In this study, an activity metabolomics screen was performed in cancer cells with tryptophan metabolites and found that 3HK was the most potent disruptor of TCA cycle metabolism and glutathione levels. Induction of IDO expression, followed by kynureninase knockdown, led to significantly increased levels of ROS, apoptosis, and cell death compared to the non-targeting control. Thus, these results suggest that inhibition of kynureninase may be an effective way to disrupt tryptophan metabolism and induce cell death in IDO-expressing cancer cells.

### Methods

### Quantitatively Mapping Amino Acid Properties QMAP)

Ingredients were bought or made in bulk and frozen in aliquots at −20° C. One day prior to cell culture, reagents were thawed in a 37° C. water bath and combined. pH was adjusted to 7.4 +/−0.05, and media was filtered using a 0.22 μm sterile bottle-top filter. Media was kept at 4° C. until time of use. Due to its sensitivity to degradation, glutamine was not added to media until time of use.

### Crystal Violet Cell Cytotoxicity Assay

HCT15 cells were plated in 1:1 DMEM: QMAP media on a clear 96-well plate. After 18 hours, cells were treated with 200 μl of QMAP (CTRL) or QMAP plus an amino acid (2 mM). Cells were then grown for 48 hours, followed by a live/dead assay using the Crystal Violet Cell Cytotoxicity Assay Kit (BioVision, #K329) according to the manufacturer's directions. Briefly, cells were washed with 200 μl of 1× washing solution and incubated in 50 μl of crystal violet staining solution for 20 minutes. Cells were washed four times with 200 μl of 1× washing solution to remove dead cells. Cells were then incubated in 100 μl of solubilization solution for 20 minutes, and absorbance was measured at 595 nm.

### Cell-TiterGlo Luminescent Cell Viability Assay

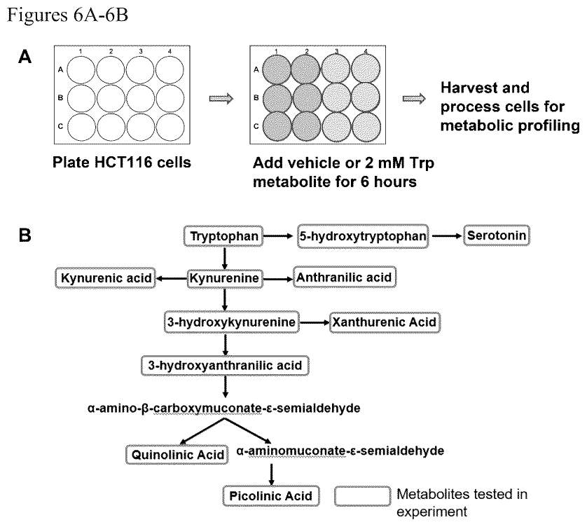

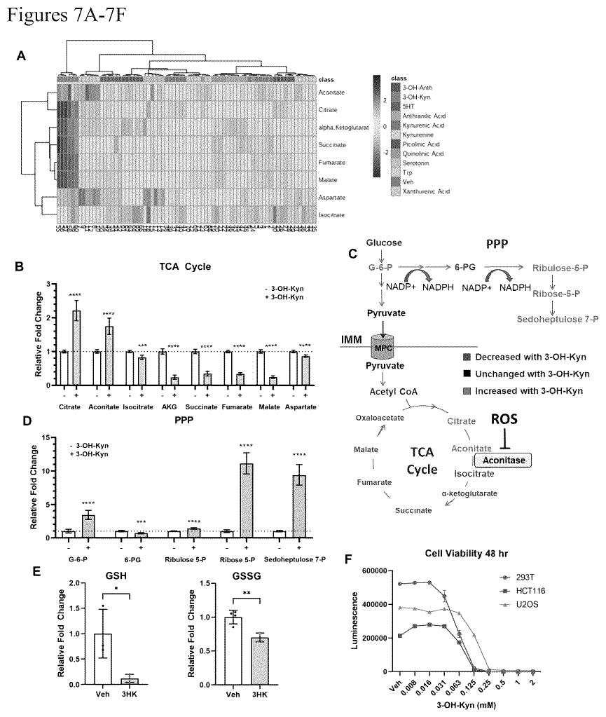

HT29, 293T, U2OS, and 3T3-LI cells were plated on an opaque 96-well plate in 1:1 DMEM. After 18 hours, cells were treated with 100 μl of DMEM (vehicle) or DMEM plus 2 mM tryptophan for 24 hours (FIGS. 6A-6B), 100 μl of QMAP (vehicle) or QMAP plus 3HK in a 2-fold dilution series for 48 hours (FIGS. 7A-7F), or QMAP (vehicle) or IFN-γ (50 ng/μL) for 24 hours, followed by KYNU siRNA or non-targeting siRNA treatment for 24 and 48 hours. At the appropriate time point, 100 μl of CellTiter-Glo reagent (Promega #G7570) was added to each well. The plate was gently mixed for 2 minutes to induce lysis, incubated at room-temperature for 10 minutes, and then luminescence was measured.

Quantitative PCR (qPCR)

Total RNA from HCT116 cells was extracted using the TRIzol (Invitrogen #15596026) method. cDNA synthesis of equal amounts of RNA from each sample was achieved using the High-Capacity cDNA Reverse Transcription Kit. SYBR Green ER SuperMix was used during qPCR. Relative abundance of mRNA was normalized to the abundance of cyclophilin mRNA. Primer sequences (5′-3′): KYNU-IF: 5;GGGGGTGCCAGCTAACAATA, KYNU-IR: TCCGCTTGTCACAAACCACT, KYNU-2F: GATCCTGTTCAGTGGGGTGC, KYNU-2R: GCCAACATAACAACCCTTCGC; Cyclophilin-F: GGAGATGGCACAGGAGGAAA, Cyclophilin-R: GCCCGTAGTGCTTCAGTTT.

siRNA Transfection

Predesigned DsiRNAs (2 nmol) targeting KYNU (Design ID: hs.Ri.KYNU.13.2, hs.Ri.KYNU.13.3), a HPRT-SI positive control DsiRNA, and a negative control DsiRNA were obtained from Integrated DNA Technologies. DsiRNAs were resuspended in nuclease-free duplex buffer. For each well of a 96-well plate, 6 pmol of DsiRNA were diluted in 100 μL Opti-MEM media and pipette-mixed. 0.3 μL/well of Lipofectamine RNAiMAX was added, pipette-mixed, and incubated at room-temperature for 20 minutes. 20 μL of diluted HCT116 cells were added to each well to achieve 30-50% confluence after 24 hours. The final concentration of RNA was 10 nM. Cells were grown at 37° C. in a CO₂ incubator until time of cell viability assay, knockdown assay, or flow cytometry.

### Annexin V and Propidium Iodide for Apoptosis and Live/Dead Staining

Cells were dissociated using Accutase (Invitrogen #00-4555-56) (Trypsin) for 20 minutes at 37° C. The single cell suspension was then washed twice in PBS-/-. 5 μL of Annexin V-Alexa488 labeling buffer was added to 100 μL of cells for 15 minutes. 5 μL of propidium iodide (PI) buffer was then added for 15 minutes. Cells were immediately run on an a 4-laser acoustic-focusing Attune NXT Flow Cytometer (ThermoFisher). Signal was read at an Ex/Em of 490/525 nm for Annexin V and 535/617 nm for PI. Data were analyzed using FlowjoX.

### DCFDA Cellular ROS Detection Assay

Cells were dissociated using Accutase (Trypsin) for 20 minutes at 37° C. The single cell suspension was then washed twice in PBS-/-. Cells were stained and resuspended with 2′,7′-dichlorofluorescin diacetate (DCFDA) (20 μM) for 30 minutes. Cells were immediately run on a 4-laser acoustic-focusing Attune NxT Flow Cytometer (ThermoFisher). Signal was read at an

Ex/Em of 485/535 nm. Data were analyzed using FlowjoX.

### Xanthommatin Synthesis

Xanthommatin was prepared. Briefly, 2 mM 3HK was mixed with 2 mM H₂O₂, and 10 pg/ml HRP in sodium phosphate buffer (50 mM, pH 7.4). The reactions were carried out at 20° C. with exposure to room air.

### Metabolomics Sample Preparation

6-well plates were treated for 6 hours with 2 mM of the indicated tryptophan metabolite for 6 hours before media was quickly removed, and plates were washed 2× with ice-cold PBS and 2× with ice-cold water before being snap frozen with liquid nitrogen. Cell plates were lyophilized overnight before metabolite extraction. To extract metabolites, 1 ml of an extraction buffer composed of 2:2:1 methanol/acetonitrile/water with internal standards at 1 μg/ml each (D4-Citric Acid, 13C5-Glutamine, 13C5-Glutamic Acid, 13C6-Lysine, 13C5-Methionine, 13C3-Serine, D4-Succinic Acid, 13C11-Tryptophan, and D8-Valine; Cambridge Isotope Laboratories) was added to the lyophilized cell samples. Cells were scraped for 20 seconds and collected into Eppendorf tubes, flash-frozen in liquid nitrogen, and sonicated for 10 minutes.

Samples were then placed on a rotating platform at −20° C. for 1 hour and centrifuged at 4° C. for 10 minutes at 21,000×g. 300 μL of the cleared metabolite extracts were transferred to fresh tubes for additional processing. An equal volume of each extract was pooled to serve as a quality control (QC) sample, which was analyzed at the beginning, end, and at a regular interval throughout an instrument run. Extraction buffer alone was analyzed as a processing blank sample. Metabolite extracts, the quality control sample, and the processing blank were evaporated to dryness using a speed-vacuum.

### GC-MS Analysis

The dried metabolite extracts, QC sample, and processing blank sample, were derivatized using methoxyamine hydrochloride (MOX) and N,O-Bis (trimethylsilyl) trifluoroacetamide (TMS) (Sigma). Briefly, dried extracts were reconstituted in 30 μL of 11.4 mg/ml MOX in anhydrous pyridine (VWR), vortexed for 10 minutes, and heated at 60° C. for 1 hour. Next, 20 μL TMS was added to each reconstituted extract, vortexed for 1 minute, and heated at 60° C. for 30 min. The derivatized metabolite extracts, blanks, and QC were immediately analyzed using GC/MS.

GC chromatographic separation was conducted on a Thermo Trace 1300 GC with a TraceGold TG-5SilMS column (0.25 μM film thickness; 0.25 mm ID; 30 m length). An injection volume of 1 μL was used for all samples, blanks, and QCs. The GC was operated in split mode with the following settings: 20:1 split ratio; split flow: 24 μL/min, purge flow: 5 mL/min, Carrier mode: Constant Flow, Carrier flow rate: 1.2 mL/min). The GC inlet temperature was 250° C. The GC oven temperature gradient was as follows: 80° C. for 3 min, ramped at 20° C./min to a maximum temperature of 280° C., which was held for 8 min. The injection syringe was washed 3 times with pyridine between each sample. Metabolites were detected using a Thermo ISQ single quadrupole mass spectrometer. The data was acquired from 3.90 to 21.00 min in EI mode (70eV) by single ion monitoring (SIM).

### LC-MS Analysis

Dried extracts were reconstituted in 30 μL of acetonitrile/water (1:1, V/V), vortexed for 10 minutes, and incubated at −20° C. for 18 hours. Samples were then centrifuged at 4° C. for 2 minutes at 21,000×g and the supernatant was transferred to autosampler vials for analysis.

For LC chromatographic separation, 2 μL of reconstituted metabolite extracts, QC sample, and processing blank were run on a Millipore SeQuant ZIC-PHILIC (2.1×150 mm, 5 um particle size, Millipore Sigma #150460) column with a ZIC-PHILIC guard column (20×2.1 mm, Millipore Sigma #150437) attached to a Thermo Vanquish Flex UHPLC. The mobile phase comprised Buffer A [20 mM (NH₄)₂CO₃, 0.1% NH4OH (v/v)] and Buffer B [acetonitrile]. The chromatographic gradient was run at a flow rate of 0.150 mL/min as follows: 0-20 min-linear gradient from 80 to 20% Buffer B; 20-20.5 min-linear gradient from 20 to 80% Buffer B; and 20.5-28 min-hold at 80% Buffer B. Data were acquired using a Thermo Q Exactive MS operated in full-scan, polarity-switching mode with a spray voltage set to 3.0 kV, the heated capillary held at 275° C., and the HESI probe held at 350° C. The sheath gas flow was set to 40 units, the auxiliary gas flow was set to 15 units, and the sweep gas flow was set to 1 unit. MS data acquisition was performed with polarity switching in a range of m/z 70-1,000, with the resolution set at 70,000, the AGC target at 10e6, and the maximum injection time at 200 ms. The QC sample was analyzed at the beginning and end of the LC-MS run and after every 10 samples.

### Mass Spectrometry and Data Analysis

Acquired LC-MS and GC-MS data were processed using the Thermo Scientific TraceFinder software. Targeted metabolites were identified by matching accurate mass and retention times to the University of Iowa Metabolomics Core Facility's in-house library of confirmed standards. After peak area integration by TraceFinder, NOREVA software was applied for signal drift correction on a metabolite-to-metabolite basis using the pooled QC sample that had been analyzed throughout the instrument run. Within individual samples, Post-NOREVA metabolite levels were divided by the total metabolite load (sum of all metabolite values) to equally weight individual metabolites for comparison across samples.

### Results

### Tryptophan Decreases Cell Proliferation in Colon Cancer Cells

An amino acid screen of all 20 essential and non-essential amino acids was performed to assess the differential effects of amino acids on cancer cell proliferation. Because commercially available cell culture media such as DMEM or RPMI contain variable concentrations of amino acids, a chemically defined, custom minimal media was designed containing all amino acids present at 0.3 mM, referred to as QMAP. HCT15 cells, a colon cancer cell line, were plated in 50:50 DMEM: QMAP and left to attach overnight. Media was then changed to 100% QMAP, and cells were treated with 2 mM of individual amino acids. After 48 hours, cell proliferation was measured by crystal violet dye, which binds to ribose-like molecules such as DNA and assumes dead cells become unattached and are washed away. Most amino acids increased cell proliferation compared to QMAP only, however, cells treated with tryptophan exhibited nearly a 50% decrease in cell viability (FIG. 5A). This suggests that, compared to other amino acids, tryptophan is uniquely cytotoxic to HCT15 cells at high concentrations.

To test whether tryptophan inhibition of growth applied to other cell lines and persisted in conventional cell culture media, two human cancer cell lines (HT29, U2OS), a transformed human embryonic kidney cell line (293T), and a mouse, fibroblast-like cell line (3T3-L1) were grown in DMEM for 24 hours and then treated with 2 mM tryptophan or vehicle. After 24 hours, cells were assayed by CellTiter-Glo, which indicates cell viability by measuring ATP production. All cell lines showed significant decreases in cell viability compared to vehicle (FIG. 5B). These data demonstrate that the cytotoxic effects of high concentration tryptophan are not exclusive to HCT15 cells or in the QMAP custom media.

### 3HK Potently Disrupts TCA Cycle Metabolism, Glutathione Levels, and Cell Viability

To better understand the metabolic effects that could be contributing to decreased cell viability, an activity metabolomics screen was performed in HCT116 cells with tryptophan and its metabolites (FIG. 6A). Cells were treated with 2 mM tryptophan metabolites (FIG. 6B) or vehicle for 6 hours and metabolic profiling was performed via gas-chromatography mass spectrometry (GC-MS). Because prior pilot studies showed evidence of tryptophan affecting TCA cycle metabolites, we examined the effects of tryptophan metabolites on the TCA cycle first and found that 3HK exhibited more potent disruption than the other tryptophan metabolites (FIG. 7A). Interestingly, proximal TCA cycle metabolites such as citrate and aconitate were significantly increased, while distal TCA cycle metabolites were drastically and significantly decreased, demonstrating a clear flux disruption between aconitate and isocitrate (FIG. 7B). Because aconitase, the enzyme that converts aconitate to isocitrate, contains an iron-sulfur cluster that is sensitive to oxidation, we hypothesized that ROS from 3HK or one its products could be inhibiting aconitase activity and preventing metabolism of citrate and aconitate (FIG. 7C). Significantly increased levels of glucose 6-phosphate, ribose 5-phosphate, and ribulose 5-phosphate indicated increased flux of carbon through the pentose phosphate shunt, which is used to produce NADPH for the reduction of the antioxidant glutathione (FIG. 7D). Accordingly, metabolic profiling of the same cell samples on LC-MS revealed significantly decreased levels of reduced and oxidized glutathione (FIG. 7E), which is a metabolic signature of ROS. Next, the dose-dependent effects of 3HK on cell viability (293T, U2OS, HCT116) was then assessed. All cell lines tested exhibited a dose-dependent decrease in cell viability as measured by CellTiter-Glo (FIG. 37F). Together, these data demonstrate that 3HK potently affects normal cellular metabolism and is likely responsible for the cytotoxic effects of high tryptophan concentrations.

### Induction of IDO Followed by Kynureninase Knockdown Increases ROS, Apoptosis, and Cell Death in Colon Cancer Cells

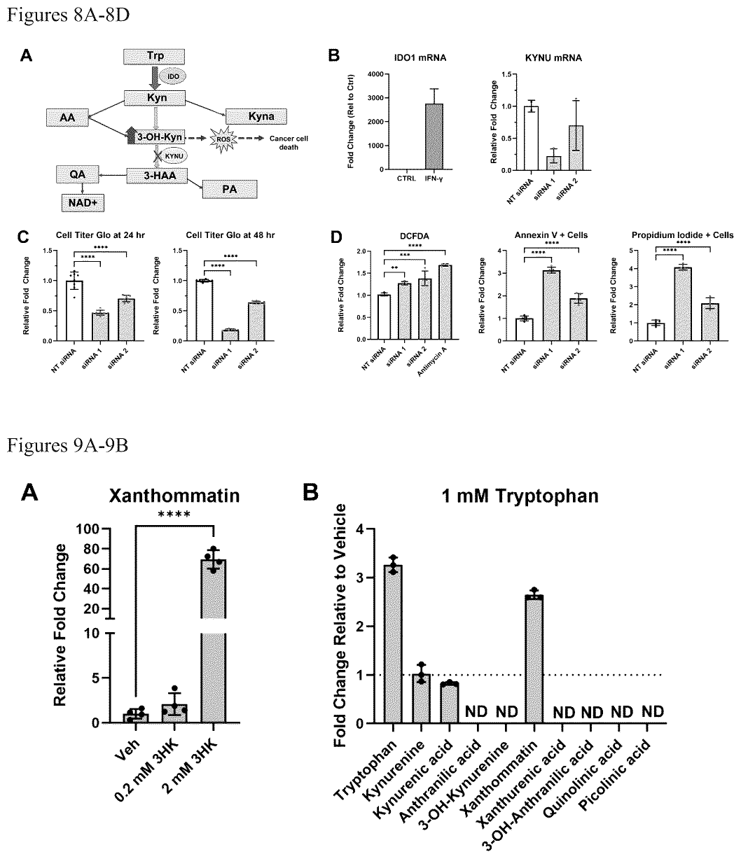

Given the detrimental effects of 3HK on cell metabolism and viability, it was hypothesized that 3HK accumulation resulting from kynureninase inhibition in IDO overexpressing cancer could result in cell toxicity and death (FIG. 8A). To test this hypothesis, IDO expression was induced in HCT116 cells using the known IDO potentiator IFN-γ (FIG. 8B).

Then, KYNU, the gene that encodes kynureninase, was knocked down using DsiRNAs and assessed cell viability at 24 and 48 hours. KYNU-knockdown decreased cell viability at both time points compared to the non-targeting control (FIG. 8C). Fluorescent staining using DCFDA, Annexin V, and propidium iodide, revealed increased ROS, apoptosis, and cell death, respectively (FIG. 8D). Furthermore, DsiRNA knockdown potency correlated to the degree of ROS, apoptosis, and cell death that was observed. This suggests that increasing 3HK levels in IDO overexpressing cancers may is sufficient to induce cell death.

The ROS-generating 3HK Metabolite Xanthommatin Increases with Tryptophan Administration

Although there is a clear association between 3HK and elevated cellular ROS, the goal was to better understand whether 3HK or one of its metabolites was producing the radical species. 3HK can spontaneously oxidize (or autoxidize) to form the radical-producing compound xanthommatin, a dimer of 3HK. It is interesting to note that xanthommatin has been well-characterized in insect eye pigmentation, however, only one study mentions xanthommatin being produced by mammalian biological material. This study showed that cytochrome c and cytochrome oxidase were able to convert 3HK to xanthommatin in rat liver mitochondria. Using a previously described xanthommatin synthesis method, xanthommatin was generated from 3-hydroxykynurenine. Xanthommatin analyzed by LC-MS/MS matched publicly available xanthommatin fragmentation data, confirming the compound's identity. HCT116 cells were treated with 0.2 or 2 mM 3-HK for 6 hours and observed a ˜70-fold increase of xanthommatin at the 2 mM dose (FIG. 9A). Finally, HCT116 cells were treated with IFN-γ (50 ng/μL) for 24 hours to induce IDO expression and then treated with tryptophan (1 mM) for 6 hours. Metabolic profiling revealed that xanthommatin was the only tryptophan metabolite to significantly increase (FIG. 9B). These data demonstrate that tryptophan is readily metabolized to xanthommatin in HCT116 cells

### Discussion

Tryptophan metabolites have been extensively studied, particularly in the context of tumor-mediated immunosuppression and neuronal protection or cytotoxicity. However, the metabolic effects of tryptophan metabolites have not been systematically assessed in a side-by-side comparison in cells before. By adding the same concentration of each tryptophan metabolite to cancer cells in a chemically defined, custom media and performing an activity metabolomics assay, it was shown that 3HK exerts the greatest metabolic disruption on TCA cycle and pentose phosphate pathway metabolites. Increased levels of proximal TCA cycle metabolites and decreased distal TCA cycle metabolites after 3HK treatment suggest a ROS-mediated inhibition of aconitase activity. Furthermore, increased levels of pentose phosphate pathway metabolites and decreased glutathione levels are a metabolic signature of excess ROS. 3HK also decreased cell viability in multiple cell lines in a dose-dependent manner.

It is interesting to note that 3HK has been the focus of relatively few tryptophan metabolite studies, with some reviews failing to mention it all. Studies that have investigated it have been predominantly limited to a neuronal context. 3-hydroxykynurenine's ROS-mediated toxicity in cancer cells in this study indicates a metabolic vulnerability that could potentially be exploited in IDO-positive tumors. Inducing IDO expression to increase tryptophan catabolismo 3HK and knocking down KYNU to decrease 3HK degradation were sufficient to induce significant apoptosis and cell death in culture. These results raise the possibility that inhibition of kynureninase in IDO-positive tumors could lead to sufficient intracellular levels of 3-hydroxykynurenine/xanthommatin to mediate cytotoxic effects. Notably, although small molecule inhibitors have been developed to target IDO and other kynurenine pathway enzymes, no inhibitors have been developed to target kynureninase.

Overall, the present investigation of the effects of tryptophan metabolites on cellular metabolism revealed a novel metabolic vulnerability to high concentrations of 3-hydroxykynurenine, which could be exploited in cancer cells by inhibiting kynureninase, the enzyme that metabolizes 3-hydroxykynurenine. It was also discovered that HCT116 cells produce xanthommatin, a 3HK metabolite that has not been described in human cells and has been shown to generate radical species in vitro. Furthermore, although 3HK was not detectable with tryptophan supplementation, xanthommatin levels were significantly increased, suggesting that xanthommatin production from 3-hydroxykynurenine may catalyze ROS formation and mediate the cytotoxicity observed in our studies.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

When used in this specification and the claims as an adverb rather than a preposition, “about” means “approximately” and comprises the stated value and every non-negative value within 10% of that value; in other words, “about 100%” includes 90% and 110% and every value in between.

Unless stated otherwise, every range or interval includes both endpoints and every value in between.

The invention has been described as “comprising” certain steps and/or elements, which those of skill in the art also “consist of” or “consist essentially of” those steps and/or elements. As used herein, the transitional term “comprising” is synonymous with “including,” “containing,” or “characterized by,” is inclusive or open-ended and does not exclude additional, unrecited elements or method steps. Where the invention is intended to be more narrowly defined, the terms “consisting of” or “consisting essentially of” also are used to describe the invention. As used herein, the transitional phrase “consisting of” excludes any element, step, or ingredient not specified in the claim. The transitional phrase “consisting essentially of” limits the scope of a claim to the specified elements or steps and those that do not materially affect the basic and novel characteristics of the claimed invention. As used herein, a claim reciting “consisting essentially of” occupies a middle ground between closed claims reciting a “consisting of” format and fully open claims that recite “comprising.”

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein.

Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A Qualitatively Mapping Amino Acid Properties (QMAP) cell culture media composition comprising:
(a) an amino acid component comprising glycine, alanine, arginine, aspartic acid, cysteine, glutamic acid, histidine, isoleucine, leucine, lysine, methionine, phenylalanine, proline, serine, threonine, tryptophan, tyrosine, valine, and optionally glutamine and/or asparagine;
wherein the amino acids are present in the component at uniform concentrations;
(b) an inorganic salt component comprising Calcium Chloride (CaCl₂ anhydrous), Magnesium Sulfate (MgSO₄ anhydrous), Potassium Chloride (KCl), Sodium Bicarbonate (NaHCO₃), Sodium Chloride (NaCl), and/or Sodium Phosphate monobasic (NaH₂PO₄—H₂O);
(c) an RPMI 1640 Vitamin Solution component comprising Biotin, Choline chloride, D-Calcium pantothenate, Folic Acid (folate), Niacinamide, Pyridoxine hydrochloride, Riboflavin, Thiamine hydrochloride, and/or Vitamin B12,; and
optionally Ammonium chloride, i-Inositol and/or Para-aminobenzoic acid (PABA);
(d) auxiliary ingredient component comprising D-Glucose, Sodium pyruvate, 3-Hydroxybutyric acid sodium salt, L-Carnitine hydrochloride, Sodium L-lactate, Penicillin-streptomycin, and/or Dialyzed fetal bovine serum. and
(e) optionally, minerals and metals.

2. The QMAP cell culture media composition of claim 1, wherein the amino acid component comprises glutamine.

3. The QMAP cell culture media composition of claim 1, wherein the amino acid component comprises asparagine.

4. The QMAP cell culture media composition of claim 1, wherein the amino acids in the amino acid component comprise L-amino acids.

5. The QMAP cell culture media composition of claim 1, wherein one of the amino acid species is labelled with a detectable label.

6. The QMAP cell culture media composition of claim 1, wherein the inorganic salt component comprises NaHCO₃.

7. The QMAP cell culture media composition of claim 1, wherein the inorganic salt component comprises CaCl₂ anhydrous present at a final concentration of 0.3 to 3.0 mM in the QMAP culture media.

8. The QMAP cell culture media composition of claim 1, wherein the inorganic salt component comprises MgSO₄ anhydrous present at a final concentration of 0.3 to 50 mM in the QMAP culture media.

9. The QMAP cell culture media composition of claim 1, wherein the inorganic salt component comprises KCl present at a final concentration of 2.0 to 20.0 mM in the QMAP culture media.

10. The QMAP cell culture media composition of claim 1, wherein the inorganic salt component comprises NaCl present at a final concentration of 60 to 160 mM in the QMAP culture media.

11. The QMAP cell culture media composition of claim 1, wherein the inorganic salt component comprises NaH₂PO₄—H₂O present at a final concentration of 0.3 to 3.0 mM in the QMAP culture media.

12. The QMAP cell culture media composition of claim 1, wherein the QMAP Cell Culture media comprises an auxiliary ingredient component, wherein the auxiliary ingredient component comprises D-glucose, L-Carnitine hydrochloride and dialyzed fetal bovine serum, and optionally comprises Sodium pyruvate, 3-Hydroxybutyric acid sodium salt, Sodium L-lactate, and/or Penicillin-streptomycin.

13. The QMAP cell culture media of claim 1, wherein the minerals or metals comprise iron, zinc, copper, selenium, molybdenum, vanadium, manganese, chromium, and/or cobalt.

14. A method for cultivating a cell in vitro, comprising:
(a) providing QMAP cell culture media of claim 1; and
(b) propagating or maintaining the cell in the QMAP cell culture media to form a cell culture.

15. A Qualitatively Mapping Amino Acid Properties (QMAP) cell culture media composition comprising:
(a) an amino acid component comprising glycine, alanine, arginine, aspartic acid, cysteine, glutamic acid, histidine, isoleucine, leucine, lysine, methionine, phenylalanine, proline, serine, threonine, tryptophan, tyrosine, valine, and optionally glutamine and/or asparagine;
wherein the amino acids are present in the component at uniform concentrations;
(b) an inorganic salt component comprising Calcium Chloride (CaCl₂ anhydrous), Magnesium Sulfate (MgSO₄ anhydrous), Potassium Chloride (KCl), Sodium Chloride (NaCl), and/or Sodium Phosphate monobasic (NaH₂PO₄—H₂O);
(c) an RPMI 1640 Vitamin Solution component comprising Biotin, Choline chloride, D-Calcium pantothenate, Folic Acid (folate), Niacinamide, Pyridoxine hydrochloride, Riboflavin, Thiamine hydrochloride, and/or Vitamin B12,; and
optionally Ammonium chloride, i-Inositol and/or Para-aminobenzoic acid (PABA);
(d) auxiliary ingredient component comprising D-Glucose, Sodium pyruvate, 3-Hydroxybutyric acid sodium salt, L-Carnitine hydrochloride, Sodium L-lactate, Penicillin-streptomycin, and/or Dialyzed fetal bovine serum. and
(e) optionally, minerals and metals.

16. A Qualitatively Mapping Amino Acid Properties (QMAP) cell culture media composition comprising:
(a) a pool of amino acids, wherein the pool of amino acids consists of:
(1) alanine, arginine, aspartic acid, cysteine, glutamic acid, histidine, isoleucine, leucine, lysine, methionine, phenylalanine, proline, serine, threonine, tryptophan, tyrosine, valine, glutamine and asparagine;
(2) glycine, arginine, aspartic acid, cysteine, glutamic acid, histidine, isoleucine, leucine, lysine, methionine, phenylalanine, proline, serine, threonine, tryptophan, tyrosine, valine, glutamine and asparagine;
(3) glycine, alanine, aspartic acid, cysteine, glutamic acid, histidine, isoleucine, leucine, lysine, methionine, phenylalanine, proline, serine, threonine, tryptophan, tyrosine, valine, glutamine and asparagine;
(4) glycine, alanine, arginine, cysteine, glutamic acid, histidine, isoleucine, leucine, lysine, methionine, phenylalanine, proline, serine, threonine, tryptophan, tyrosine, valine, glutamine and asparagine;
(5) glycine, alanine, arginine, aspartic acid, glutamic acid, histidine, isoleucine, leucine, lysine, methionine, phenylalanine, proline, serine, threonine, tryptophan, tyrosine, valine, glutamine and asparagine;
(6) glycine, alanine, arginine, aspartic acid, cysteine, histidine, isoleucine, leucine, lysine, methionine, phenylalanine, proline, serine, threonine, tryptophan, tyrosine, valine, glutamine and asparagine;
(7) glycine, alanine, arginine, aspartic acid, cysteine, glutamic acid, isoleucine, leucine, lysine, methionine, phenylalanine, proline, serine, threonine, tryptophan, tyrosine, valine, glutamine and asparagine;
(8) glycine, alanine, arginine, aspartic acid, cysteine, glutamic acid, histidine, leucine, lysine, methionine, phenylalanine, proline, serine, threonine, tryptophan, tyrosine, valine, glutamine and asparagine;
(9) glycine, alanine, arginine, aspartic acid, cysteine, glutamic acid, histidine, isoleucine, lysine, methionine, phenylalanine, proline, serine, threonine, tryptophan, tyrosine, valine, glutamine and asparagine;
(10) glycine, alanine, arginine, aspartic acid, cysteine, glutamic acid, histidine, isoleucine, leucine, methionine, phenylalanine, proline, serine, threonine, tryptophan, tyrosine, valine, glutamine and asparagine;
(11) glycine, alanine, arginine, aspartic acid, cysteine, glutamic acid, histidine, isoleucine, leucine, lysine, phenylalanine, proline, serine, threonine, tryptophan, tyrosine, valine, glutamine and asparagine;
(12) glycine, alanine, arginine, aspartic acid, cysteine, glutamic acid, histidine, isoleucine, leucine, lysine, methionine, proline, serine, threonine, tryptophan, tyrosine, valine, glutamine and asparagine;
(13) glycine, alanine, arginine, aspartic acid, cysteine, glutamic acid, histidine, isoleucine, leucine, lysine, methionine, phenylalanine, serine, threonine, tryptophan, tyrosine, valine, glutamine and asparagine;
(14) glycine, alanine, arginine, aspartic acid, cysteine, glutamic acid, histidine, isoleucine, leucine, lysine, methionine, phenylalanine, proline, threonine, tryptophan, tyrosine, valine, glutamine and asparagine;
(15) glycine, alanine, arginine, aspartic acid, cysteine, glutamic acid, histidine, isoleucine, leucine, lysine, methionine, phenylalanine, proline, serine, tryptophan, tyrosine, valine, glutamine and asparagine;
(16) glycine, alanine, arginine, aspartic acid, cysteine, glutamic acid, histidine, isoleucine, leucine, lysine, methionine, phenylalanine, proline, serine, threonine, tyrosine, valine, glutamine and asparagine;
(17) glycine, alanine, arginine, aspartic acid, cysteine, glutamic acid, histidine, isoleucine, leucine, lysine, methionine, phenylalanine, proline, serine, threonine, tryptophan, valine, glutamine and asparagine;
(18) glycine, alanine, arginine, aspartic acid, cysteine, glutamic acid, histidine, isoleucine, leucine, lysine, methionine, phenylalanine, proline, serine, threonine, tryptophan, tyrosine, glutamine and asparagine;
(19) glycine, alanine, arginine, aspartic acid, cysteine, glutamic acid, histidine, isoleucine, leucine, lysine, methionine, phenylalanine, proline, serine, threonine, tryptophan, tyrosine, valine, and asparagine;
(20) glycine, alanine, arginine, aspartic acid, cysteine, glutamic acid, histidine, isoleucine, leucine, lysine, methionine, phenylalanine, proline, serine, threonine, tryptophan, tyrosine, valine, and glutamine;
wherein the amino acids are present in the pool at uniform concentrations;
(b) an inorganic salt component comprising Calcium Chloride (CaCl₂ anhydrous), Magnesium Sulfate (MgSO⁴ anhydrous), Potassium Chloride (KCl), Sodium Bicarbonate (NaHCO₃), Sodium Chloride (NaCl), and/or Sodium Phosphate monobasic (NaH₂PO₄—H₂O);
(c) an RPMI 1640 Vitamin Solution component comprising Biotin, Choline chloride, D-Calcium pantothenate, Folic Acid (folate), Niacinamide, Pyridoxine hydrochloride, Riboflavin, Thiamine hydrochloride, and/or Vitamin B12,; and
optionally Ammonium chloride, i-Inositol and/or Para-aminobenzoic acid (PABA);
(d) auxiliary ingredient component comprising D-Glucose, Sodium pyruvate, 3-Hydroxybutyric acid sodium salt, L-Carnitine hydrochloride, Sodium L-lactate, Penicillin-streptomycin, and/or Dialyzed fetal bovine serum, and
(e) optionally, minerals and metals.
